(19) United States
(12) Patent Application Publication
(10) Pub. No.: US 2004/0152904 A1
(43) Pub. Date: Aug. 5, 2004

(54) Delta1 pyrrolines

(76) Inventors: Andrew Plant, Berkshire (GB); Fritz Maurer, Monheim (DE); Albrecht Marhold, Leverkusen (DE); Christoph Erdelen, Leichlingen (DE); Andreas Turberg, Haan (DE); Olaf Hansen, Leichlingen (DE); Angelika Lubos-Erdelen, Leichlingen (DE)

Correspondence Address: Bayer CropScience, Patent Department, 100 Bayer Road, Pittsburgh, PA, 15205-9741, US

(21) Appl. No.: 10/472,270

(22) PCT Filed: Mar. 12, 2002
(86) PCT No.: PCT/EP02/02684

(30) Foreign Application Priority Data
Mar. 22, 2001 (DE) 10113965.9

(51) Int. Cl. C 07D 4 3/02; A61K 31/41

(52) U.S. Cl. 548/253; 514/381; 548/254

(57) ABSTRACT

The invention relates to novel Δ¹-pyrrolines of the formula (I)

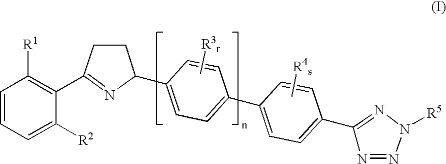

in which R¹, R², R³, R⁴, R⁵, n, r and s have the meanings given in the description, and to a plurality of processes for preparing these substances, to their use for controlling pests, and to novel intermediates and processes for their preparation.

The present invention relates to novel Δ¹-pyrrolines, to a plurality of processes for their preparation and to their use as pesticides.

It is already known that numerous Δ¹-pyrrolines have insecticidal properties (cf. WO 00/21958, WO 99/59968, WO 99/59967 and WO 98/22438). The activity of these substances is good; however, in some cases it is unsatisfactory.

This invention now provides novel A -pyrrolines of the formula (I)

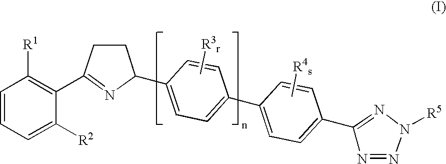

in which

R¹ represents halogen or methyl,

R² represents hydrogen or halogen,

R³ and R⁴ independently of one another represent halogen or represent in each case optionally substituted alkyl or alkoxy,

R⁵ represents hydrogen, alkylcarbonyl or represents in each case optionally substituted alkyl, alkylsulphonyl or 1-methyl-cycloalkyl,

n represents 0 or 1,

r and s independently of one another represent 0, 1 or 2.

Depending on the type and number of substituents, the compounds of the formula (I) can, if appropriate, be present as geometrical and/or optical isomers, regioisomers and/or tautomers or isomer mixtures thereof in varying compositions. What is claimed by the invention are both the pure isomers and the isomer mixtures.

Furthermore, it has been found that the novel compounds of the formula (I) can be obtained by one of the processes described below.

Δ¹-Pyrrolines of the formula (I)

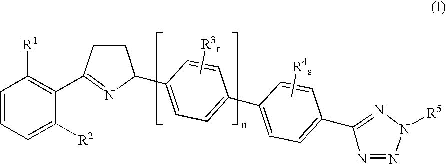

in which

R¹, R², R³, R⁴, R⁵, n, r and s have the meanings given above, can be prepared by

A) reacting Δ¹-pyrrolines of the formula (I-a)

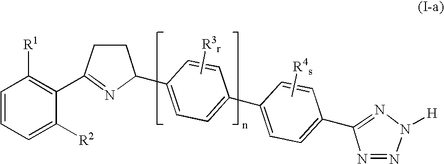

in which

R¹, R², R³, R⁴, n, r and s have the meanings given above,

A1) with a reagent of the formula (II)

R⁵⁻¹—Z (II)

in whch

R⁵⁻¹ represents alkylcarbonyl or represents in each case optionally substituted alkyl or alkylsulphonyl,

Z represents a leaving group,

if appropriate in the presence of a diluent and if appropriate in the presence of an acid binder or

A2) with an alcohol of the formula (III)

R⁵⁻²—OH (III)

in which

R⁵⁻² represents in each case optionally substituted tertiary alkyl or 1-methyl-cycloalkyl,

in the presence of a strong acid.

Δ¹-Pyrrolines of the formula (I-b)

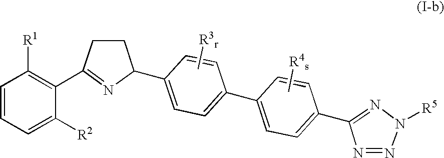

in which

R¹, R², R³, R⁴, R⁵, r and s have the meanings given above, can be prepared by

B) reacting Δ¹-pyrrolines of the formula (IV)

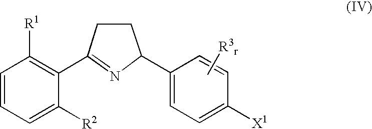

in which

R¹, R², R³ and r have the meanings given above and

X¹ represents chlorine, bromine, iodine, —OSO₂CF₃ or —OSO₂(CF₂)₃CF₃, with boron compounds of the formula (V)

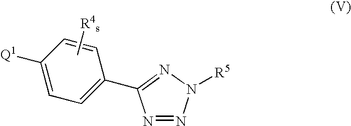

in which

R⁴, R⁵and s have the meanings given above and

Q¹ represents —B(OH)₂, (4,4,5,5-tetramethyl- 1,3,2-dioxaborolan)-2-yl, (5,5-dimethyl- 1,3,2-dioxaborinan)-2-yl, (4,4,6-trimethyl- 1,3,2dioxaborinan)-2-yl or 1,3,2-benzodioxaborol-2-yl,

in the presence of a catalyst, if appropriate in the presence of an acid binder and if appropriate in the presence of a diluent, or

C) by reacting Δ¹-pyrrolines of the formula (VI)

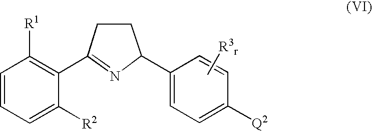

in which

R¹, R², R³ and r have the meanings given above and

Q² represent —B(OH)₂, (4,4,5,5-tetramethyl-1,3,2-dioxaborolan)-2-yl, (5,5-dimethyl-1,3,2-dioxaborinan)-2-yl, (4,4,6-trimethyl-1,3,2dioxaborinan)-2-yl or 1,3,2-benzodioxaborol-2-yl,

with phenyltetrazoles of the formula (VII)

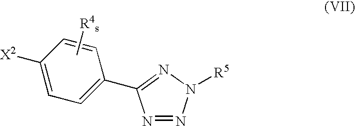

in which

R⁴, R⁵ and s have the meanings given above and

X² represents chlorine, bromine, iodine, —OSO₂CF₃ or —OSO₂(CF₂)₃CF₃,

in the presence of a catalyst, if appropriate in the presence of an acid binder and if appropriate in the presence of a diluent, or

D) by reacting Δ¹-pyrrolines of the formula (IV) (IV)

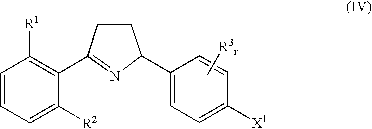

in which

R¹, R², R³, r and X¹ have the meanings given above,

with phenyltetrazoles of the formula (VII)

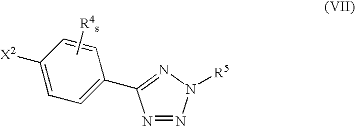

in which

R⁴, R⁵, s and X² have the meanings given above, in the presence of a catalyst, in the presence of a diboronic acid ester, if appropriate in the presence of an acid binder and if appropriate in the presence of a diluent in a tandem reaction.

Finally, it has been found that the compounds of the formula (I) according to the invention have very good insecticidal properties and can be used both in crop protection and in the protection of materials for controlling undesirable pests, such as insects.

The formula (I) provides a general definition of the Δ¹-pyrrolines according to the invention.

R¹ preferably represents halogen or methyl.

R² preferably represents hydrogen or halogen.

R³ and R⁴ independently of one another preferably represent halogen, alkyl, halogenoalkyl, alkoxy or halogenoalkoxy.

R⁵ preferably represents hydrogen, alkylcarbonyl, alkyl, halogenoalkyl, alkoxyalkyl, alkoxycarbonylalkyl, alkylsulphonyl, halogenoalkylsulphonyl or 1-methyl-cycloalkyl, which may optionally be morio- to trisubstituted by alkyl.

n preferably represents 0 or 1.

r and s independently of one another preferably represent 0, 1 or 2.

R¹ particularly preferably represents fluorine, chlorine or methyl.

R² particularly preferably represents hydrogen, fluorine or chlorine.

R³ and R⁴ independently of one another particularly preferably represent fluorine, chlorine, bromine, C₁-C₆-alkyl, C₁-C₆-halogenoalkyl, C₁-C₆-alkoxy or C₁-C₆halogenoalkoxy.

R⁵ particularly preferably represents hydrogen, C₁-C₆-aikylcarbonyl, C₁-C₈-alkyl, C₁-C₆-halogenoalkyl, C₁-C₆-alkoxy-C₁-C₆-alkyl, C₁-C₆-alkoxycarbonyl-C₁-C₆-alkyl, C₁-C₆-alkylsulphonyl, C₁-C₆-halogenoalkylsulphonyl or 1-methyl-C₃-C₆-cycloalkyl, which may be mono- to trisubstituted by C₁-C₄-alkyl.

n particularly preferably represents 0 or 1.

r and s independently of one another particularly preferably represent 0, 1 or 2.

R¹ very particularly preferably represents fluorine, chlorine or methyl.

R² very particularly preferably represents hydrogen, fluorine or chlorine.

R³ and R⁴ independently of one another very particularly preferably represent fluorine, chlorine, C₁-C₄-alkyl, C₁-C₄-halogenoalkyl having 1 to 9 fluorine, chlorine and/or bromine atoms, C₁-C₄-alkoxy or C₁-C₄-halogenoalkoxy having 1 to 9 fluorine, chlorine and/or bromine atoms.

R⁵ very particularly preferably represents hydrogen, C₁-C₄-alkylcarbonyl, C₁-C₈alkyl, C₁-C₆-halogenoalkyl having 1 to 13 fluorine, chlorine and/or bromine atoms, C₁-C₄-alkoxy-C₁-C₄-alkyl, C₁-C₄-alkoxycarbonyl-C₁-C₄-alkyl, C₁-C₄alkylsulphonyl, C₁-C₄-halogenoalkylsulphonyl having 1 to 9 fluorine, chlorine and/or bromine atoms, 1-methyl-C₃-C₆-cycloalkyl.

n very particularly preferably represents 0 or 1.

r and s independently of one another very particularly preferably represent 0, 1 or 2.

R¹ especially preferably represents fluorine or chlorine.

R² especially preferably represents hydrogen or fluorine.

R³ and R⁴ independently of one another especially preferably represent fluorine, chlorine, methyl, ethyl, n-propyl, isopropyl, n-butyl, isobutyl, sec-butyl, tertbutyl, trifluoromethyl, trifluoroethyl, methoxy, ethoxy, n-propoxy, isopropoxy, n-butoxy, isobutoxy, sec-butoxy, tert-butoxy, trifluoromethoxy or trifluoroethoxy.

R⁵ especially preferably represents hydrogen, methylcarbonyl, ethylcarbonyl, n-propylcarbonyl, isopropylcarbonyl, n-butylcarbonyl, isobutylcarbonyl, secbutylcarbonyl, tert-butylcarbonyl, methyl, ethyl, n-propyl, isopropyl, n-butyl, isobutyl, sec-butyl, tert-butyl, pentyl, hexyl, heptyl, octyl, difluoromethyl, trifluoromethyl, trifluoroethyl, nonafluorobutyl, —CH₂OMe, —CH₂OEt, —CH₂O(t-Bu), —CH₂CO₂Me, —CH₂CO₂Et, —CH₂CO₂(n-Pr), —CH₂CO₂(i-Pr), —CH₂CO₂(s-Bu), —CH₂CO₂(i-Bu), —CH₂CO₂(t-Bu), —SO₂Me, —SO₂Et, —SO₂(n-Pr), —SO₂(i-Pr), —SO₂(t-Bu), —SO₂CF₃, —SO₂(CF₂)₃CF₃ or 1-methylcyclohexyl.

n especially preferably represents 0 or 1.

r and s independently of one another especially preferably represent 0, 1 or 2.

Very particular preference is furthermore given to compounds of the formula (I-b)

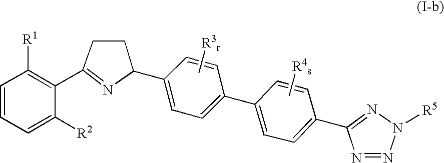

in which

R¹, R², R³, R⁴, R⁵, r and s have the meanings given above.

Very particular preference is furthermore given to compounds of the formula (I-c)

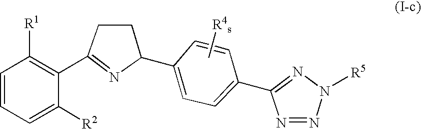

in which

R¹, R², R⁴, R⁵ and s have the meanings given above.

Very particular preference is furthermore given to compounds of the formula (I-d) having R-configuration in the 5-position of the pyrroline ring

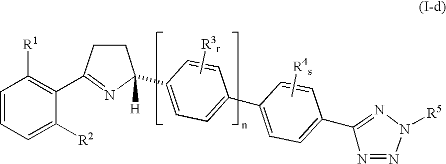

in which

R¹, R², R³, R⁴, R⁵, r and s have the meanings given above.

Very particular preference is furthermore given to compounds of the formula (I-e) having R-configuration.in the 5-position of the pyrroline ring

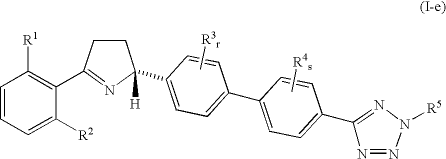

in which

R¹, R², R³, R⁴, R⁵, r and s have the meanings given above.

Very particular preference is furthermore given to compounds of the formula (I-f) having R-configuration in the 5-position of the pyrroline ring

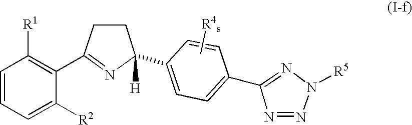

in which

R¹, R², R⁴, R⁵ and s have the meanings given above.

In the compounds of the formulae (I-b), (I-c), (I-d), (I-e) and (I-f), R¹, R², R³, R⁴, R⁵, n, r and s preferably, particularly preferably, very particularly preferably and especially preferably have the meanings which have already been mentioned above as being preferred, particularly preferred, etc., for these radicals.

Saturated hydrocarbon radicals such as alkyl can in each case be straight-chain or branched as far as this is possible, including in combination with heteroatoms, such as, for example, in alkoxy. Hexyl, for example, may represent 3-methyl-pentan-3-yl.

However, the abovementioned general or preferred radical definitions or illustrations can also be combined with one another as desired, i.e. between the respective ranges and preferred ranges. They apply both to the end products and, correspondingly, to the precursors and intermediates.

Using 5-{4-[5-(2,6-difluorophenyl)-3,4-dihydro-2H-pyrrol-2-yl]phenyl }-2H-tetrazole and n-propyl bromide as starting materials, the course of the process (A1) according to the invention can be illustrated by the equation below.

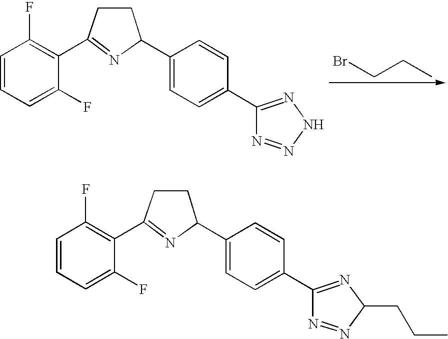

Using 5-{4-[5-(2,6-difluorophenyl)-3,4-dihydro-2H-pyrrol-2-yl]phenyl}-2H-tetrazole and 3-methyl-pentan-3-ol as starting materials, the course of the process (A2) according to the invention can be. illustrated by the equation below.

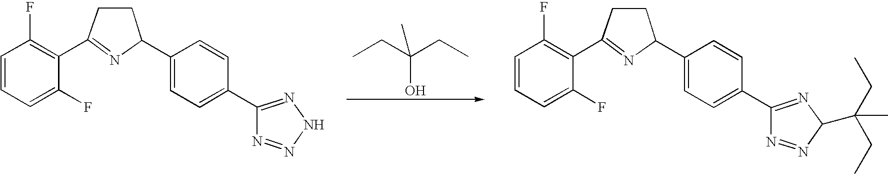

Using 4-[5-(2,6-difluorophenyl)-3,4-dihydro-2H-pyrrol-2-yl]phenyltrifluoromethanesulphonate and 4-(2-tert-butyl-2H-tetrazol-5-yl)phenylboronic acid as starting materials and a palladium catalyst, the course of the process (B) according to the invention can be illustrated by the equation below.

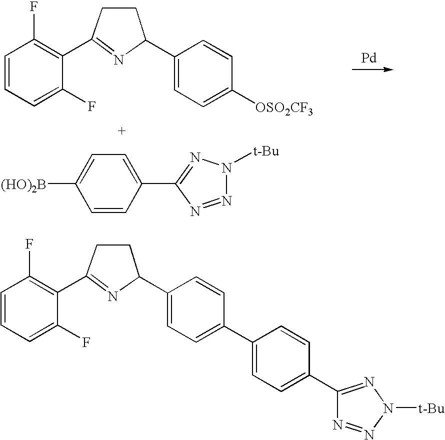

Using 5-(2,6-difluorophenyl)-2-[4-(4,4,5,5-tetramethyl-1,3,2-dioxaborolan-2yl)phenyl]-3,4-dihydro-2H-pyrrole and 5-(4-bromophenyl)-2-tert-butyl-2H-tetrazole as starting materials and a palladium catalyst, the course of the process (C) according to the invention can be illustrated by the equation below.

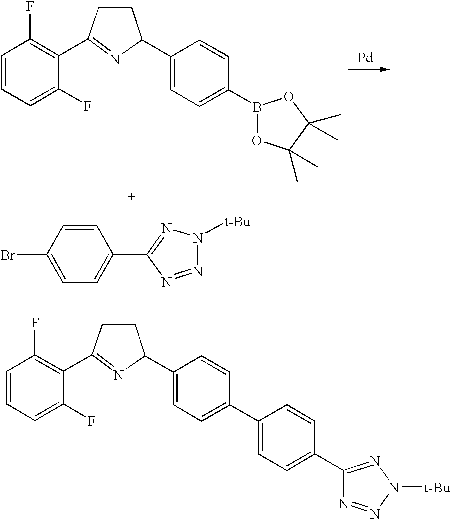

Using 4-[5-(2,6-difluorophenyl)-3,4-dihydro-2H-pyrrol-2-yl]phenyltrifluoromethanesulphonate, 5-(4-bromophenyl)-2-tert-butyl-2H-tetrazole as starting materials, 4,4,4′,4′,5,5,5′,5′-octamethyl-2,2′-bis-1,3,2-dioxaborolane as diboronic acid ester and a palladium catalyst, the course of the process (D) according to the invention can be illustrated by the equation below.

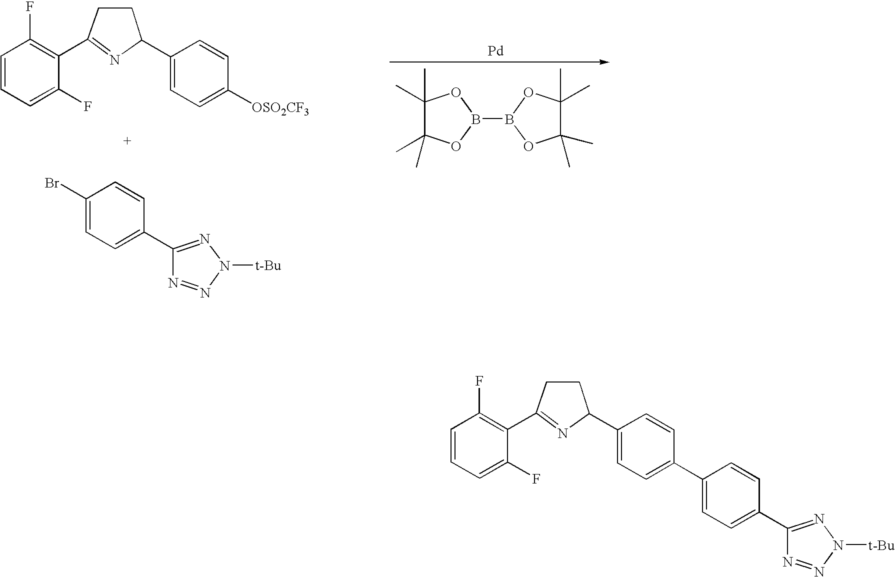

Explanation of the Processes and Intermediates

Process (A)

The formula (I-a) provides a general definition of the Δ¹-pyrrolines required as starting materials for carrying out the process (A) according to the invention. In this formula, R¹, R², R³, R⁴, n, r and s preferably, particularly preferably, very particularly preferably and especially preferably have those meanings which have already been mentioned in connection with the description of the compounds of the formula (I) according to the invention as being preferred, particularly preferred, etc., for these radicals.

Δ¹-Pyrrolines of the formula (I-a) are likewise compounds according to the invention. They can be prepared by

a) reacting nitrites of the formula (VIII)

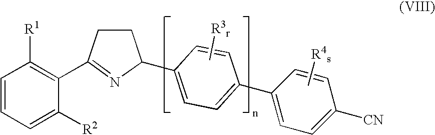

in which

R¹, R², R³, R⁴, n, r and s have the meanings given above,

with an azide of the formula (IX)

G—N₃ (IX)

in which

G represents Na, SiMe₃ or SnMe₃,

if appropriate in the presence of a solvent (for example toluene).

The formula (VIII) provides a general definition of the nitriles required as starting materials for carrying out the process (a). In this formula, R¹, R², R³, R⁴, n, r and s preferably, particularly preferably, very particularly preferably and especially preferably have those meanings which have already been mentioned in connection with the description of the compounds of the formula (I) according to the invention as being preferred, particularly preferred, etc., for this radical.

Nitriles of the formula (VIII) are novel. They can be prepared, for example, by

b) reacting aminoketones of the formula (X)

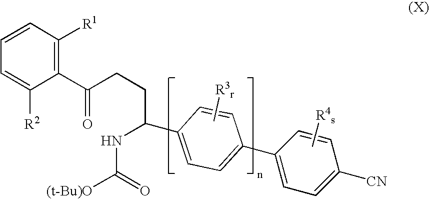

in which

R¹, R², R³, R⁴, n, r and s have the meanings given above, with a Lewis acid or a protonic acid (for example trifluoroacetic acid), if appropriate in the presence of a diluent (for example dichloromethane).

The formula (IX) provides a general definition of the azides required as starting materials for carrying out the process (a). In this formula, G preferably represents SiMe₃ or SnMe₃.

Azides of the formula (IX) are known.

The formula (X) provides a general definition of the aminoketones required as starting materials for carrying out the process (b). In this formula, R¹, R², R³, R⁴, n, r and s preferably, particularly preferably, very particularly preferably and especially preferably have those meanings which have already been mentioned in connection with the description of the compounds of the formula (I) according to the invention as being preferred, particularly preferred, etc., for this radical.

Aminoketones of the formula (X) are novel. They can be prepared by

c) reacting lactams of the formula (XI)

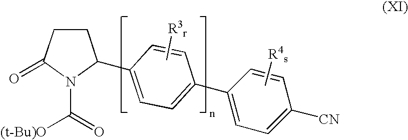

in which

R³, R⁴, n, r and s have the meanings given above,

with metallated aromatic compounds of the formula (XII)

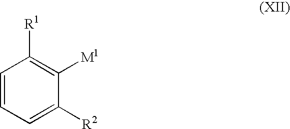

in which

R¹ and R² have the meanings given above and

M¹ represents Li, MgCl, MgBr, MgI or ZnCl,

at temperatures between −70° C. and +70° C., if appropriate in the presence of a diluent (for example tetrahydrofuran).

The formula (XI) provides a general definition of the lactams required as starting materials for carrying out the process (c). In this formula, R³, R⁴, n, r and s preferably, particularly preferably, very particularly preferably and especially preferably have those meanings which have already been mentioned in connection with the description of the compounds of the formula (I) according to the invention as being preferred, particularly preferred, etc., for this radical.

Lactams of the formula (XI) are novel. They can be prepared by

d) reacting lactarns of the formula (XIII)

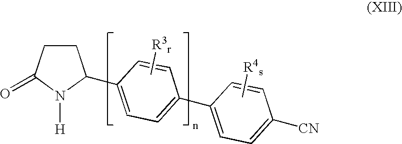

in which

R³, R⁴, n, r and s have the meanings given above,

with, for example, di-tert-butyl dicarbonate in the presence of a base (for example dimethylaminopyridine) and, if appropriate, in the presence of a diluent (for example dichloromethane) (cf. Tetrahedron Lett. 1998, 39, 2705-2706).

The formula (XII) provides a general definition of the metallated aromatic compounds required as starting materials for carrying out the process (c). In this formula, R¹ and R² preferably, particularly preferably, very particularly preferably and especially preferably have those meanings. which have already been mentioned in connection with the description of the compounds of the formula (I) according to the invention as being preferred, particularly preferred, etc., for these radicals. M¹ preferably represents Li, MgCl, MgBr, MgI or ZnCl, particularly preferably Li, MgCl, MgBr or MgI, very particularly preferably Li, MgCl or MgBr.

Metallated aromatic compounds of the formula (XII) are known and/or can be prepared by known methods (for example lithiation or Grignard reaction) from the corresponding aromatic or halogenated aromatic compounds.

The formula (XIII) provides a general definition of the lactams required as starting materials for carrying out the process (d). In this formula, R³, R⁴, n, r and s preferably, particularly preferably, very particularly preferably and especially preferably have those meanings which have already been mentioned in connection with the description of the substances of the formula (I) according to the invention as being preferred, particularly preferred, etc., for these radicals.

Lactams of the formula (XIII) are novel.

Lactams of the formula (XIII-a)

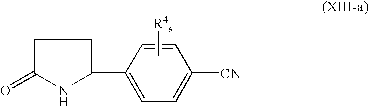

in which

R⁴ and s have the meanings given above,

can be prepared, for example, by

e) reacting lactams of the formula (XIV)

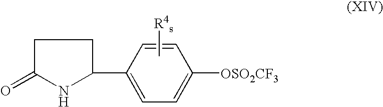

in which

R⁴ and s have the meanings given above,

with a metal cyanide from the group consisting of sodium cyanide, potassium cyanide and zinc(II) cyanide

in the presence of a catalyst [for example Pd(PPh₃)₄] and, if appropriate, in the presence of a diluent (for example dimethylformamide) (cf. Syn. Commun. 1995, 25, 3255).

Lactams of the formula (XIII-b)

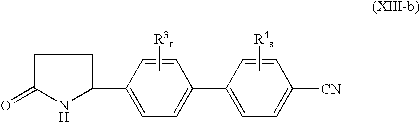

in which

R³, R⁴, r and s have the meanings given above,

can be prepared, for example, by

f) reacting 5-ethoxy-2-pyrrolidinone with biphenyls of the formula (XV)

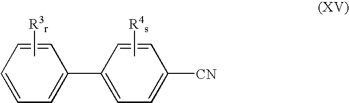

in which

R³, R⁴, r and s have the meanings given above,

in the presence of hydrofluoric acid and, if appropriate, in the presence of a diluent (for example dichloromethane).

The formula (XIV) provides a general definition of the lactams required as starting materials for carrying out the process (e). In this formula, R⁴ and s preferably, particularly preferably, very particularly preferably and especially preferably have those meanings which have already been mentioned in connection with the description of the substances of the formula (I) according to the invention as being preferred, particularly preferred, etc., for these radicals.

Lactams of the formula (XIV) are known and/or can be prepared by known processes (cf. WO 98/22438).

The formula (XV) provides a general definition of the biphenyls required as starting materials for carrying out the process (f). In this formula, R³, R⁴, r and s preferably, particularly preferably, very particularly preferably and especially preferably have those meanings which have already been mentioned in connection with the description of the substances of the formula (I) according to the invention as being preferred, particularly preferred, etc., for these radicals.

Biphenyls of the formula (XV) are known.

The formula (II) provides a general definition of the reagents required as starting materials for carrying out the process (A1) according to the invention. In this formula, R⁵⁻¹ preferably represents alkylcarbonyl, alkyl, halogenoalkyl, alkoxyalkyl, alkoxycarbonylalkyl, alkylsulphonyl or halogenoalkylsulphonyl. R⁵⁻¹ particularly preferably represents C₁-C₆-alkylcarbonyl, C₁-C₈-alkyl, C₁-C₆-halogenoalkyl, C₁-C₆alkoxy-C₁-C₆-alkyl, C₁-C₆-alkoxycarbonyl-C₁-C₆-alkyl, C₁-C₆-alkylsulphonyl or C₁-C₆-halogenoalkylsulphonyl. R⁵⁻¹ very particularly preferably represents C₁-C₄alkylcarbonyl, C₁-C₈-alkyl, C₁-C₆-halogenoalkyl having 1 to 13 fluorine, chlorine and/or bromine atoms, C₁-C₄-alkoxy-C₁-C₄-alkyl, C₁-C₄-alkoxycarbonyl-C₁-C₄-alkyl, C₁-C₄-alkylsulphonyl or C₁-C₄-halogenoalkylsulphonyl having 1 to 9 fluorine, chlorine and/or bromine atoms. R⁵⁻¹ especially preferably represents methylcarbonyl, ethylcarbonyl, n-propylcarbonyl, isopropylcarbonyl, n-butylcarbonyl, isobutylcarbonyl, sec-butylcarbonyl, tert-butylcarbonyl, methyl, ethyl, n-propyl, isopropyl, n-butyl, isobutyl, sec-butyl, tert-butyl, pentyl, hexyl, heptyl, octyl, difluoromethyl, trifluoromethyl, trifluoroethyl, nonafluorobutyl, —CH₂OMe, —CH₂OEt, —CH₂O(t-Bu), —CH₂CO₂Me, —CH₂CO₂Et, —CH₂CO₂(n-Pr), —CH₂CO₂(i-Pr), —CH₂CO₂(s-Bu), —CH₂CO₂(i-Bu), —CH₂CO₂(t-Bu), —SO₂Me, —SO₂Et, —SO₂(n-Pr), —SO₂(i-Pr), —SO₂(t-Bu), —SO₂CF₃, —SO₂(CF₂)₃CF₃. Z preferably represents halogen, hydroxyl, alkylcarbonyloxy. Z particularly preferably represents chlorine, bromine, iodine, hydroxyl, C₁-C₄-alkylcarbonyloxy. Z very particularly preferably represents chlorine, bromine, iodine, hydroxyl, methylcarbonyloxy. Z especially preferably represents chlorine, bromine, iodine, hydroxyl.

Reagents of the formula (II) are known.

The formula (III) provides a general definition of the alcohols required as starting materials for carrying out the process (A2) according to the invention. In this formula, R⁵⁻² preferably represents tertiary alkyl, tertiary halogenoalkyl or 1-methylcycloalkyl, which may optionally be mono- to trisubstituted by alkyl. R⁵⁻² particularly preferably represents tertiary C₁-C₈-alkyl, tertiary C₁-C₆-halogenoalkyl or 1-methyl-C₃-C₆-cycloalkyl, which may optionally be mono- to trisubstituted by C₁-C₄-alkyl. R⁵⁻² very particularly preferably represents tertiary C₁-C₈-alkyl, tertiary C₁-C₅halogenoalkyl having 1 to 11 fluorine, chlorine and/or bromine atoms, 1-methyl-C₃-C₆-cycloalkyl. R⁵⁻² especially preferably represents tert-butyl, tert-pentyl, 3-methylpentan-3-yl, 3-ethyl-pentan-3-yl or 1-methylcyclohexyl.

Reagents of the formula (III) are known.

Suitable diluents for carrying out the process (A1) according to the invention are all customary inert organic solvents. Preference is given to using optionally halogenated aliphatic, alicyclic or aromatic hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene or o-dichlorobenzene; ethers, such as diethyl ether, dibutyl ether, glycol dimethyl ether, diglycol dimethyl ether, tetrahydrofuran or dioxane; ketones, such as acetone, methyl ethyl ketone, methyl isopropyl ketone or methyl isobutyl ketone; esters, such as methyl acetate or ethyl acetate; nitriles, such as acetonitrile or propionitrile; amides, such as, for example, N,N-dimethylformamide, N,N-dimethylacetamide or N-methylpyrrolidone; and also dimethyl sulphoxide, tetramethylene sulphone or hexamethylphosphoric triamide.

Suitable acid acceptors for carrying out the process (A1) according to the invention are all acid binders which are customarily used for such reactions. Preference is given to using alkali metal and alkaline earth metal hydrides, such as lithium hydride, sodium hydride, potassium hydride or calcium hydride; alkali metal and alkaline earth metal hydroxides, such as lithium hydroxide, sodium hydroxide, potassium hydroxide or calcium hydroxide; alkali metal and alkaline earth metal carbonates or bicarbonates, such as sodium carbonate or potassium carbonate or sodium bicarbonate or potassium bicarbonate or calcium carbonate; alkali metal acetates, such as sodium acetate or potassium acetate, alkali metal alkoxides, such as sodium tert-butoxide or potassium tert-butoxide; furthermore basic nitrogen compounds, such as trimethylamine, tripropylamine, tributylamine, diisobutylamine, dicyclohexylamine, ethyldiisopropylamine, ethyldicyclohexylamine, N,Ndimethylbenzylamine, N,N-dimethylaniline, pyridine, 2-methyl-, 3-methyl-, 4methyl-, 2,4-dimethyl-, 2,6-dimethyl-, 2-ethyl-, 4-ethyl- and 5-ethyl-2methylpyridine, 1,5-diazabicyclo[4.3.0]-non-5-ene (DBN), 1,8-diaza-bicyclo[5.4.0]undec-7-ene (DBU), 1,4-diazabicyclo[2.2.2]-octane (DABCO).

Suitable acids for carrying out the process (A2) according to the invention are all protonic acids which are customarily used for such reactions. Preference is given to using sulphuric acid or trifluoroacetic acid or mixtures of these two.

When carrying out the process (A) according to the invention, the reaction temperatures can be varied within a relatively wide range. In general, the reaction is carried out at temperatures between 0° C. and 100° C., preferably at temperatures between 10° C. and 80° C.

When carrying out the process (A1) according to the invention, in general 1 mol or a slight excess of the compound of the formula (II). is employed per mole of the compound of the formula (I-a) However, it is also possible to employ the reaction components in other ratios. Work-up is carried out by customary methods. In general, the reaction mixture is partitioned between two liquid phases and the organic phase is separated off, dried and concentrated under reduced pressure. The resulting crude products are, if appropriate, freed of any impurities that may still be present using customary methods, such as chromatography or recrystallization.

When carrying out the process (A2) according to the invention, in general from 1 to 8 mol, preferably from 1 to 3 mol, of the compound of the formula (III) and from 1 to 3 mol, preferably from 1 to 2 mol, of acid are employed per mole of the compound of the formula (I-a). However, it is also possible to employ the reaction components in other ratios. Work-up is carried out by customary methods. In general, the reaction mixture is partitioned between two liquid phases and the organic phase is separated off, dried and concentrated under reduced pressure. The resulting crude products are, if appropriate, freed from any impurities that may still be present using customary methods such as chromatography or recrystallization.

Process (B)

The formula (IV) provides a general definition of the Δ¹-pyrrolines required as starting materials for carrying out the process (B) according to the invention. In this formula, R¹, R², R³ and r preferably, particularly preferably, very particularly preferably and especially preferably have those meanings which have already been mentioned in connection with the description of the compounds of the formula (I) according to the invention as being preferred, particularly preferred, etc. for these radicals. X¹ preferably represents bromine, iodine, —OSO₂CF₃ or —OSO₂(CF₂)₃CF₃, particularly preferably bromine, —OSO₂CF₃ or —OSO₂(CF₂)₃CF₃, very particularly preferably bromine or —OSO₂CF₃.

Δ¹-Pyrrolines of the formula (IV) can be prepared by known processes (cf. WO 98/22438).

The formula (V) provides a general definition of the boron compounds required as starting materials for carrying out the process (B) according to the invention. In this formula, R⁴, R⁵ and s preferably, particularly preferably, very particularly preferably and especially preferably have those meanings which have already been mentioned in connection with the description of the substances of the formula (I) according to the invention as being preferred, particularly preferred, etc., for these radicals. Q¹ preferably represents —B(OH)₂, (4,4,5,5-tetramethyl-1,3,2-dioxaborolan)-2-yl, (5,5dimethyl-1,3,2-dioxaborinan)-2-yl, (4,4,6-trimethyl-1,3,2-dioxaborinan)-2-yl or 1,3,2-benzodioxaborol-2-yl, particularly preferably —B(OH)₂, (4,4,5,5-tetramethyl-1,3,2-dioxaborolan)-2-yl, (5,5-dimethyl-1,3,2-dioxaborinan)-2-yl or (4,4,6-trimethyl-1,3,2-dioxaborinan)-2-yl, very particularly preferably (4,4,5,5-tetramethyl-1,3,2dioxaborolan)-2-yl, (5,5-dimethyl-1,3,2-dioxaborinan)-2-yl.

Some of the boron compounds of the formula (V) are known (cf. WO 96/16946, WO 93/10106 and US 5 130 439). They can be prepared, for example, by

g) reacting phenyltetrazoles of the formula (VII),

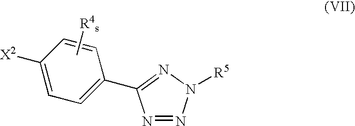

in which

R⁴, R⁵ and s have the meanings given above and

X² represents chlorine, bromine, iodine, —OSO₂CF₃ or —OSO₂(CF₂)₃CF₃,

with boric acid esters in the presence of a metallating agent (for example butyllithium) or with a diboronic acid ester in the presence of a catalyst, if appropriate in the presence of an acid binder and if appropriate in the presence of a diluent (cf. J. Org. Chem. 1995, 60, 7508; Tetrahedron Lett. 1997, 38, 3447).

Phenyltetrazoles of the formula (VII) are described in detail under process (C).

When carrying out the process (B) according to the invention, in general 1 mol or a slight excess of a compound of the formula (V) and from 0.5 to 5 mol % of a palladium catalyst are employed per mole of the compound of the formula (IV). However, it is also possible to employ the reaction components in other ratios. Workup is carried out by customary methods. In general, the reaction mixture is taken up in ethyl acetate and the organic phase is washed with water, dried over sodium sulphate, filtered and concentrated. The residue is, if appropriate, freed from any impurities that may still be present using customary methods, such as chromatography or recrystallization.

Process (C)

The formula (VI) provides a general definition of the Δ¹-pyrrolines required as starting materials for carrying out the process (C) according to the invention. In this formula, R¹, R², R³ and r preferably, particularly preferably, very particularly preferably and especially preferably have those meanings which have already been mentioned in connection with the description of the substances of the formula (I) according to the invention as being preferred, particularly preferred, etc., for these radicals. Q² preferably represents (4,4,5,5-tetramethyl-1,3,2-dioxaborolan)-2-yl, (5,5dimethyl-1,3,2-dioxaborinan)-2-yl, (4,4,6-trimethyl-1,3,2-dioxaborinan)-2-yl or 1,3,2-benzodioxaborol-2-yl, particularly preferably (4,4,5,5-tetramethyl-1,3,2dioxaborolan)-2-yl, (5,5-dimethyl-1,3,2-dioxaborinan)-2-yl or (4,4,6-trimethyl-1,3,2dioxaborinan)-2-yl, very particularly preferably (4,4,5,5-tetramethyl-1,3,2-dioxaborolan)-2-yl, (5,5-dimethyl-1,3,2-dioxaborinan)-2-yl.

Δ¹-Pyrrolines of the formula (VI) can be prepared, for example, by

h) reacting compounds of the formula (IV)

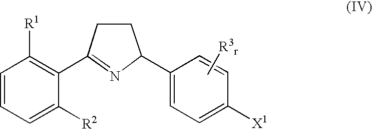

in which

R¹, R², R³, r and X¹ have the meanings given above, with a diboronic acid ester in the presence of a catalyst, if appropriate in the presence of an acid binder and if appropriate in the presence of a diluent (cf. J. Org. Chem. 1995, 60, 7508; Tetrahedron Lett. 1997, 38, 3447).

The compounds of the formula (IV) required as starting materials for the process (h) have already been described under the explanation of process (B).

Suitable diboronic acid esters for carrying out the process (h) are 4,4,4′,4′,5,5,5′,5′-octamethyl-2,2′-bis-1,3,2-dioxaborolane, 5,5,5′,5′-tetramethyl-2,2′-bis-1,3,2dioxaborinane, 4,4,4′,4′,6,6′-hexamethyl-2,2′-bis-1,3,2-dioxaborinane or 2,2′-bis1,3,2-benzodioxaborol. Preference is given to using 4,4,4′,4′,5,5,5′,5′-octamethyl-2,2′-bis-1,3,2-dioxaborolane, 5,5,5′,5′-tetramethyl-2,2′-bis-1,3,2-dioxaborinane or 4,4,4′,4′,6,6′-hexamethyl-2,2′-bis-1,3,2-dioxaborinane, particularly preferably 4,4,4′,4′,5,5,5′,5′-octamethyl-2,2′-bis-1,3,2-dioxaborolane or 5,5,5′,5′-tetramethyl-2,2′bis-1,3,2-dioxaborinane, very particularly preferably 4,4,4′,4′,5,5,5′,5′-octamethyl-2,2′-bis-1,3,2-dioxaborolane. The formula (VII) provides a general definition of the phenyltetrazoles required as starting materials for carrying out the process (C) according to the invention. In this formula, R⁴, R⁵ and s preferably, particularly preferably, very particularly preferably and especially preferably have those meanings which have already been mentioned in connection with the description of the substances of the formula (I) according to the invention as being preferred, particularly preferred, etc., for these radicals. X² preferably represents bromine, chlorine, iodine or —OSO₂CF₃, particularly preferably bromine, chlorine or iodine, very particularly preferably bromine.

Some of the phenyltetrazoles of the formula -(V-II)- are known (cf., --for example, EP 0339549, J. Med. Chem. 1991, 34, 1125-1136). They can be prepared, for example, by

i) reacting phenyltetrazoles of the formula (VII-a)

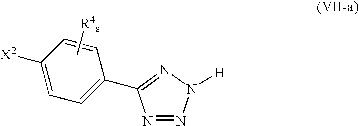

in which

X², R⁴ and s have the meanings given above,

i1) with a reagent of the formula (II)

R⁵⁻¹—Z (II)

in which

R⁵⁻¹ and Z have the meanings given above,

if appropriate in the presence of a diluent (for example acetonitrile) and if appropriate in the presence of an acid binder (for example potassium carbonate) or

i2) with an alcohol of the formula (III)

R⁵⁻² OH (III)

in which

R⁵⁻² has the meanings given above,

in the presence of a strong acid (for example sulphuric acid, trifluoroacetic acid or mixtures of the two).

The phenyltetrazoles of the formula (VII-a) are known and/or can be prepared from the corresponding benzonitriles according to known processes.

Starting materials of the formulae (II) and (III) have already been described under the explanation of process (A).

When carrying out the process (C) according to the invention, in general 1 mol or a slight excess of a compound of the formula (VII) and from 0.5 to 5 mol % of a palladium catalyst are employed per mole of the compound of the formula (VI). However, it is also possible to employ the reaction components in other ratios. Workup is carried out by customary methods. In general, the reaction mixture is taken up in ethyl acetate and the organic phase is washed with water, dried over sodium sulphate, filtered and concentrated. The residue is, if appropriate, freed from any impurities that may still be present using customary methods, such as chromatography or recrystallization.

Process (D)

In a first reaction step, a compound of the formula (IV) is coupled with a diboronic acid ester in the presence of a palladium catalyst, if appropriate in the presence of an acid binder and if appropriate in the presence of a solvent. Without any isolation of the intermediate, a compound of the formula (VII) is coupled in the same reaction vessel in a second reaction step in the presence of a catalyst, if appropriate in the presence of an acid binder and if appropriate in the presence of a solvent (cf., for example, Tetrahedron Lett. 1997, 38, 3841).

The process (D) according to the invention can be carried out in two variants. It is possible either to initially charge a compound of the formula (IV) or to initially charge a compound of the formula (VII). Process (D) is to be considered a tandem reaction of the processes (B) and (C) described above.

The Δ¹-pyrrolines of the formula (IV) required as starting materials for carrying out the process (D) according to the invention have already been described under the explanation of process (B).

The phenyltetrazoles of the formula (VII) required as starting materials for carrying out the process (D) according to the invention have already been described under the explanation of process (C).

Diboronic acid esters suitable for carrying out the process (D) according to the invention have already been mentioned under the description of process (h).

When carrying out the process (D) according to the invention, in general 1 mol or a slight excess of a diboronic ester and 1 mol or a slight excess of a compound of the formula (VII), and from 0.5 to 5 mol %, preferably 3 mol %, of a palladium catalyst are employed per mole of the compound of the formula (IV). However, it is also possible to employ the reaction components in other ratios. It is possible to initially charge the compound of the formula (IV) or, alternatively, the compound of the formula (VII). Work-up is carried out by customary methods. In general, the reaction mixture is diluted with water and extracted with ethyl acetate. The organic phase is washed, dried, filtered and concentrated The residue is, if appropriate, freed from any impurities that may still be present using customary methods, such as chromatography or recrystallization.

When carrying out the processes (B), (C) and (D) according to the invention, in each case a palladium catalyst is employed, which for its part can be used with or without addition of further ligands. The catalyst used is preferably PdCI₂(dppf) [dppf=1,1′-bis(diphenylphosphino)ferrocene], Pd(PPh₃)₄, PdCl₂(PPh₃)₂, PdCl₂(CH₃CN)₂, Pd₂(dba)₃ [dba=dibenzylideneacetone] or Pd(OAc)₂, particularly preferably PdCl₂(dppf), Pd(PPh₃)₄, PdCl₂(PPh₃)₂ or Pd(OAc)₂, very particularly preferably PdCl₂(dppf) or PdCl₂(PPh₃)₂.

Suitable ligands are triarylphosphines, trialkylphosphines or arsines. Preference is given to using dppf, PPh₃, P(t-Bu)₃, Pcy₃ or AsPh₃, particularly preferably dppf.

Suitable diluents for carrying out the processes (B), (C) and (D) according to the invention are in each case all customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decaline; chlorobenzene, dichlorobenzene, dichloromethane, chloroform, tetrachloromethane, dichlorethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or arisole; nitriles, such as acetonitrile, propionitrile, n- or isobutyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide, or sulphones, such as sulpholane. Particular preference is given to using acetone, dimethoxyethane, dioxane, tetrahydrofuran, dimethylformamide, dimethylacetamide, dimethyl sulphoxide, ethanol, toluene or, if appropriate, mixtures of the diluents mentioned with water.

Suitable acid binders for carrying out the processes (B), (C) and (D) according to the invention are in each case all inorganic and organic bases which are customary for such reactions. Preference is given to using alkaline earth metal or alkali metal hydroxides, such as sodium hydroxide, calcium hydroxide, potassium hydroxide, or else ammonium hydroxide, alkali metal carbonates, such as sodium carbonate, potassium carbonate, potassium bicarbonate, sodium bicarbonate, alkali metal or alkaline earth metal acetates, such as sodium acetate, potassium acetate, calcium acetate, alkali metal fluorides, and also tertiary amines, such as trimethylamine, triethylamine, tributylamine, N,N-dimethylaniline, pyridine, N-methylpiperidine, N,N-dimethylaminopyridine, diazabicyclooctane (DABCO), diazabicyclononene (DBN) or diazabicycloundecene (DBU). However, it is also possible to operate without additional acid binder, or to employ an excess of the amine component, so that it simultaneously acts as acid binder. Barium hydroxide, sodium hydroxide, potassium hydroxide, tripotassium phosphate, caesium carbonate, potassium carbonate, sodium carbonate, potassium acetate, triethylamine, potassium tertbutoxide, caesium fluoride or potassium fluoride are used with particular preference.

When carrying out the process (B), (C) and (D) according to the invention, the reaction temperatures can in each case be varied within a relatively wide range. In general, the reactions are carried out at temperatures between 0° C. and 140° C., preferably between 20° C. and 120° C., particularly preferably between 60° C. and 100° C.

Chiral Compounds of the Formula (I-d)

To prepare chiral compounds of the formula (I-d), it is possible, for example, to subject Δ¹-pyrrolines of the formula (I-g)

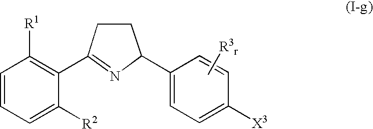

in which

R¹, R², R³ and r have the meanings given above and

X³ represents chlorine, bromine, iodine or cyano,

to an optical resolution. To this end, for example, methods of preparative chromatography, preferably the High Performance Liquid Chromatography (HPLC) method, are employed. Here, a chiral stationary silica gel phase is used. A tris(3,5-dimethylphenylcarbamate)-cellulose-modified silica gel has been found to be particularly suitable for separating the compounds of the formula (I-g) into the two enantiomers. This separating material is commercially available. However, it is also possible to use other stationary phases. Suitable mobile phases are all customary inert organic solvents, and mixtures of these. Preference is given to using optionally halogenated aliphatic, alicyclic or aromatic hydrocarbons, such as petroleum ether, hexane, heptane, cyclohexane; dichloromethane, chloroform; alcohols, such as methanol, ethanol, propanol; nitrites, such as acetonitrile; esters, such as methyl acetate or ethyl acetate. Particular preference is given to using aliphatic hydrocarbons, such as hexane or heptane, and alcohols, such as methanol or propanol, very particularly preferably n-heptane and isopropanol or mixtures of these. In general, the separation is carried out at temperatures between 10° C. and 60° C., preferably between 10° C. and 40° C., particularly preferably at room temperature.

Δ¹-Pyrrolines of the formula (I-g) are known and/or can be prepared by known processes (cf. WO 98/22438 for the case where X³ represents chlorine, bromine or iodine). Al-Pyrrolines of the formula (I-g) can be prepared according to process (b) if X³ represents cyano.

The (R)-configured enantiomers obtained in this manner are then used as starting materials for the processes (A), (B) or (D).

All processes according to the invention are generally carried out under atmospheric pressure. However, in each case it is also possible to operate under elevated or reduced pressure.

The active compounds according to the invention are suitable for controlling animal pests, in particular insects, arachnids and nematodes, which are encountered in agriculture, in forestry, in the protection of stored products and of materials, and in the hygiene sector, and have good plant tolerance and favourable toxicity to warm-blooded animals. They may be preferably employed as plant protection agents. They are active against normally sensitive and resistant species and against all or some stages of development. The abovementioned pests include:

From the order of the Isopoda, for example, Oniscus-asellus, Armadillidium vulgare and Porcellio scaber. From the order of the Diplopoda, for example, Blaniulus guttulatus. From the order of the Chilopoda, for example, Geophilus carpophagus and Scutigera spp. From the order of the Symphyla, for example, Scutigerella immaculata. From the order of the Thysanura, for example, Lepisma saccharina. From the order of the Collembola, for example, Onychiurus armatus. From the order. of the Orthoptera, for example, Acheta domesticus, Gryllotalpa spp., Locusta migratoria migratorioides, Melanoplus spp. and Schistocerca gregaria. From the order of the Blattaria, for example, Blatta orientalis, Periplaneta americana, Leucophaea maderae, Blattella germanica. From the order of the Dermaptera, for example, Forficula auricularia. From the order of the Isoptera, for example, Reticulitermes spp. From the order of the Phthiraptera, for example, Pediculus humanus corporis, Haematopinus spp., Linognathus spp., Trichodectes spp. and Damalinia spp. From the order of the Thysanoptera, for example, Hercinothrips femoralis, Thrips tabaci, Thrips palmi and Frankliniella accidentalis. From the order of the Heteroptera, for example, Eurygaster spp., Dysdercus intermedius, Piesma quadrata, Cimex lectularius, Rhodnius prolixus and Triatoma spp. From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp. From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura funiferana, Clysia ambiguella, Homona magnanima, Tortrix viridana, Cnaphalocerus spp., Oulema oryzae. From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus. From the order of the Hymenoptera, for example, Diprion spp., Hoplocampa spp., Lasius spp., Monomorium pharaonis and Vespa spp. From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp. From the order of the Siphonaptera, for example, Xenopsylla cheopis and Ceratophyllus spp. From the class of the Arachnida, for example, Scorpio maurus, Latrodectus mactans, Acarus siro, Argas spp., Ornithodoros spp., Dermanyssus gallinae, Eriopbyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hernitarsonemus spp., Brevipalpus spp.

The phytoparasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., xiphinema spp., Trichodorus spp., Bursaphelenchus spp.

In particular, the compounds of the formula (I) according to the invention have excellent activity against caterpillars, beetle larvae, spider mites, aphids and leafmining flies.

If appropriate, the compounds according to the invention can, at certain concentrations or application rates, also be used as herbicides or microbicides, for example as fungicides, antimycotics and bactericides. If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on the surroundings, environment or storage space by the customary treatment methods, for example by immersion spraying, evaporation, fogging, scattering, painting on and, in the case of propagation material, in particular in the case of seeds, also by applying one or more coats.

The active compounds according to the invention can be converted into the customary formulations, such as solutions, emulsions, wettable powders, suspensions, powders, dusts, pastes, soluble powders, granules, suspension-emulsion concentrates, natural and synthetic materials impregnated with active compound and microencapsulations in polymeric substances.

These formulations are produced in a known manner, for example by mixing the active compounds according to the invention with extenders, that is liquid solvents and/or solid carriers, optionally with the use of surfactants, that is emulsifiers and/or dispersants and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

As solid carriers there are suitable: for example ammonium salts and ground natural minerals such as kaolins, clays, talc, chalk, quartz, attapulgite, montmorillonite or diatomaceous earth, and ground synthetic minerals, such as highly disperse silica, alumina and silicates; as solid carriers for granules there are suitable: for example crushed and fractionated natural rocks such as calcite, marble, pumice, sepiolite and dolomite, and also synthetic granules of inorganic and organic meals, and granules of organic material such as sawdust, coconut shells, maize cobs-and tobacco stalks;

as emulsifiers and/or foam-formers there are suitable: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates;

as dispersants there are suitable: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate,. as well as natural phospholipids such as cephalins and lecithins, and synthetic phospholipids, can be used in the formulations. Other additives can be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compound according to the invention can be used in unmodified form or in their formulations as a mixture with other active compounds which are also known, such as insecticides, attractants, sterilizing agents, bactericides, acaricides, nematicides, fungicides, growth-regulating substances or herbicides, for example to widen the activity spectrum or to prevent the development of resistance. In many cases, synergistic effects are obtained, i.e. the activity of the mixture exceeds the activity of the individual components. The insecticides include, for example, phosphoric acid esters, carbamates, carboxylates, chlorinated hydrocarbons, phenylureas and substances produced by microorganisms,inter alia. Suitable co-components are, for example, the following compounds:

Fungicides:

aldimorph, ampropylfos, ampropylfos-potassium, andoprim, anilazine, azaconazole, azoxystrobin, benalaxyl, benodanil, benomyl, benzamacril, benzamacryl-isobutyl, bialaphos, binapacryl, biphenyl, bitertanol, blasticidin-S, bromuconazole, bupirimate, buthiobate, calcium polysulphide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon, edifenphos, epoxiconazole, etaconazole, ethirimol, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulphamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole, imazalil, imibenconazole, iminoctadine, iminoctadine albesilate, iminoctadine triacetate, iodocarb, ipconazole, iprobenfos (IBP), iprodione, irumamycin, isoprothiolane, isovaledione, kasugamycin, kresoxim-methyl, copper preparations, such as: copper hydroxide, copper naphthenate, copper oxychloride, copper sulphate, copper oxide, oxine-copper and Bordeaux mixture, mancopper, mancozeb, maneb, meferimzone; mepanipyrim, mepronil, metalaxyl; metconazole, methasulfocarb, methfuroxain, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur, quinconazole, quintozene (PCNB), sulphur and sulphur preparations, tebuconazole, tecloftalam, tecnazene, tetcyclasis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole, uniconazole, validamycin A, vinclozolin, viniconazole, zarilamide, zineb, ziram and also Dagger G, OK-8705, OK-8801, α-(1,1-dimethylethyl)-β-(2-phenoxyethyl)-1H-1,2,4-triazole-1-ethanol, α-(2,4-dichlorophenyl)-β-fluoro-β-propyl-1H-1,2,4-triazole-1-ethanol, α-(2,4-dichlorophenyl)-β-methoxy-α-methyl-1H-1,2,4-triazole-1-ethanol, α-(5-methyl-1,3-dioxan-5-yl)-β-[[4-(trifluoromethyl)-phenyl]-methylene]-1H-1,2,4triazole-1-ethanol, (5RS,6RS)-6-hydroxy-2,2,7,7-tetramethyl-5-(1H-1,2,4-triazol-1-yl)-3-octanone, (E)-α-(methoxyimino)-N-methyl-2-phenoxy-phenylacetamide, 1-isopropyl {2-methyl-1-[[[1-(4-methylphenyl)-ethyl]-amino]carbonyl]-propyl} carbamate, 1-(2,4-dichlorophenyl)-2-(1H-1,2,4-triazol-1-yl)-ethanone-O-(phenylmethyl)-oxime, 1-(2-methyl-1-naphthalenyl)-1H-pyrrole-2,5-dione, 1-(3,5-dichlorophenyl)-3-(2-propenyl)-2,5-pyrrolidinedione, 1-[(diiodomethyl)-sulphonyl]-4-methyl-benzene, [[2-(2,4-dichlorophenyl)-1,3-dioxolan-2-yl]-methyl]-1H-imidazole, 1-[[2-(4-chlorophenyl)-3-phenyloxiranyl]-methyl]-1H-1,2,4-triazole, 1-[1-[2-[(2,4-dichlorophenyl)-methoxy]-phenyl]-ethenyl]-1H-imidazole, 1-methyl-5-nonyl-2-(phenylmethyl)-3-pyrrolidinole, 2′,6′-dibromo-2-methyl-4′-trifluoromethoxy-4′-trifluoro-methyl-1,3-thiazole-5carboxanilide, 2,2-dichloro-N-[ 1-(4-chlorophenyl)-ethyl]-1-ethyl-3-methyl-cyclopropanecarboxamide, 2,6-dichloro-5-(methylthio)-4-pyrimidinyl-thiocyanate, 2,6-dichloro-N-(4-trifluoromethylbenzyl)-benzamide, 2,6-dichloro-N-[[4-(trifluoromethyl)-phenyl]-methyl]-benzamide, 2-(2,3,3-triiodo-2-propenyl)-2H-tetrazole, 2-[(1-methylethyl)-sulphonyl]-5-(trichloromethyl)-1,3,4-thiadiazole, 2-[[6-deoxy-4-O-(4-O-methyl-β-D-glycopyranosyl)-α-D-glucopyranosyl]-amino]-4methoxy-1H-pyrrolo[2,3-d]pyrimidine-5-carbonitrile, 2-aminobutane, 2-bromo-2-(bromomethyl)-pentanedinitrile, 2-chloro-N-(2,3-dihydro-1,1,3-trimethyl-1H-inden-4-yl)-3-pyridinecarboxamide, 2-chloro-N-(2,6-dimethylphenyl)-N-(isothiocyanatomethyl)-acetamide, 2-phenylphenol (OPP), 3,4-dichloro-1-[4-(difluoromethoxy)-phenyl]-1H-pyrrole-2,5-dione, 3 ,5-dichloro-N-[cyano[(1-methyl-2-propynyl)-oxy]-methyl]-benzamide, 3-(1,1-dimethylpropyl-1-oxo-1H-indene-2-carbonitrile, 3-[2-(4-chlorophenyl)-5-ethoxy-3-isoxazolidinyl]-pyridine, 4-chloro-2-cyano-N,N-dimethyl-5-(4-methylphenyl)-1H-imidazole-1-sulphonamide, 4-methyl-tetrazolo[ 1,5-a]quinazolin-5(4H)-one, 8-(1,1-dimethylethyl)-N-ethyl-N-propyl-1,4-dioxaspiro[4.5]decane-2-methanamine, 8-hydroxyquinoline sulphate, 9H-xanthene-2-[(phenylamino)-carbonyl]-9-carboxylic hydrazide, bis-(1-methylethyl)-3-methyl-4-[(3-methylbenzoyl)-oxy]-2,5-thiophenedicarboxylate, cis-1-(4-chlorophenyl)-2-(1-H-1,2,4-triazol-1-yl)-cycloheptanol, cis4-[3-[4-(1,1-dimethylpropyl)-phenyl-2-methylpropyl]-2,6-dimethyl-morpholine hydrochloride, ethyl [(4-chlorophenyl)-azo]-cyanoacetate, potassium bicarbonate, methanetetrathiol-sodium salt, methyl 1-(2,3-dihydro-2,2-dirnethyl-1H-inden-1-yl)-1H-imidazole-5-carboxylate, methyl N-(2,6-dimethylphenyl)-N-(5-isoxazolylcarbonyl)-DL-alaninate, methyl N-(chloroacetyl)-N-(2,6-dimethylphenyl)-DL-alaninate, N-(2,3-dichloro4-hydroxyphenyl)-1-methyl-cyclohexanecarboxamide, N-(2,6-dimethylphenyl)-2-methoxy-N-(tetrahydro-2-oxo-3-furanyl)-acetamide, N-(2,6-dimethylphenyl)-2-methoxy-N-(tetrahydro-2-oxo-3-thienyl)-acetamide, N-(2-chloro-4-nitrophenyl)-4-methyl-3-nitro-benzenesulphonamide, N-(4-cyclohexylphenyl)-1,4,5,6-tetrahydro-2-pyrimidinamine, N-(4-hexylphenyl)-1,4,5,6-tetrahydro-2-pyrimidinamine, N-(5-chloro-2-methylphenyl)-2-methoxy-N-(2-oxo-3-oxazolidinyl)-acetamide, N-(6-methoxy-3-pyridinyl)-cyclopropanecarboxamide, N-[2,2,2-trichloro-1-[(chloroacetyl)-amino]-ethyl]-benzamide, N-[3-chloro-4,5-bis(2-propinyloxy)-phenyl]-N′-methoxy-methanimidamide, N-formyl-N-hydroxy-DL-alanine-sodium salt, O,O-diethyl [2-(dipropylamino)-2-oxoethyl]-ethylphosphoramidothioate, O-methyl S-phenyl phenylpropylphosphoramidothioate, S-methyl 1,2,3-benzothiadiazole-7-carbothioate, spiro[2H]-1-benzopyran-2,1′(3′H)-isobenzofuran]-3′-one, 4-[(3,4-dimethoxyphenyl)-3-(4-fluorophenyl)-acryloyl]-morpholine

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:

abamectin, acephate, acetamiprid, acrinathrin, alanycarb, aldicarb, aldoxycarb, alphacypermethrin, alphamethrin, amitraz, avermectin, AZ 60541, azadirachtin, azamethiphos, azinphos A, azinphos M, azocyclotin, Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben, cadusafos, carbaryl, carbofuran, carbophenothion, carbosulfhan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine, deltamethrin, demeton M, demeton S, demeton-S-methyl, diafenthiuron, diazinon, dichlorvos, dicofol, diflubenzuron, dimethoate, dimethylvinphos, diofenolan, disulfoton, docusat-sodium, dofenapyn, eflusilanate, emamectin, empenthrin, endosulfan, Entomopfthora spp., esfenvalerate, ethiofencarb, ethion, ethoprophos, etofenprox, etoxazole, etrimfos, fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb, granulosis viruses, halofenozide, HCH, heptenophos, hexaflumuron, hexythiazox, hydroprene, imidacloprid, indoxacarb, isazofos, isofenphos, isoxathion, ivermectin, nuclear polyhedrosis viruses, lambda-cyhalothrin, lufenuron, malathion, mecarbam, metaldehyde, methamidophos, Metharhizium anisopliae, Metharhizium flavoviride, methidathion, methiocarb, methoprene, methomyl, methoxyfenozide, metolcarb, metoxadiazone, mevinphos, milbemectin, milbemycin, monocrotophos, naled, nitenpyram, nithiazine, novaluron, omethoate, oxamyl, oxydemethon M, Paecilomyces fumosoroseus, parathion A, parathion M, permethrin, phenthoate, phorate, phosalone, phosmet, phosphamidon, phoxim, pirimicarb, pirimiphos A, pirimiphos M, profenofos, promecarb, propargite, propoxur, prothiofos, prothoate, pymetrozine, pyraclofos, pyresmethrin, pyrethrum, pyridaben, pyridathion, pyrimidifen, pyriproxyfen, quinalphos, ribavirin, salithion, sebufos, silafluofen, spinosad, spirodiclofen, sulphotep, sulprofos, tau-fluvalinate, tebufenozide, tebufenpyrad, tebupirimiphos, teflubenzuron, tefluthrin, temephos, temivinphos, terbufos, tetrachlorvinphos, tetradifon, thetacypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb, vamidothion, vaniliprole, Verticillium lecanii, YI 5302,zeta-cypermethrin, zolaprofos, (1R-cis)-[5-(phenylmethyl)-3-furanyl]-methyl-3-[(dihydro-2-oxo-3(2H)furanylidene)-methyl]-2,2-dimethylcyclopropanecarboxylate, (3-phenoxyphenyl)-methyl-2,2,3 ,3-tetramethylcyclopropanecarboxylate, 1-[(2-chloro-5-thiazolyl)methyl]tetrahydro-3,5-dimethyl-N-nitro-1,3,5-triazine2(1H)-imine, 2-(2-chloro-6-fluorophenyl)-4-[4-(1,1-dimethylethyl)phenyl]-4,5-dihydro-oxazole, 2-(acetyloxy)-3-dodecyl-1,4-naphthalenedione, 2-chloro-N-[[[4-(1-phenylethoxy)-phenyl]-amino]-carbonyl]-benzamide, 2-chloro-N-[[[4-(2,2-dichloro-1,1-difluoroethoxy)-phenyl]-amino]-carbonyl]benzamide, 3-methylphenyl propylcarbamate. 4-[4-(4-ethoxyphenyl)-4-methylpentyl]-1-fluoro-2-phenoxy-benzene, 4-chloro-2-(1,1-dimethylethyl)-5-[[2-(2,6-dimethyl-4-phenoxyphenoxy)ethyl]thio] 3(2H)-pyridazinone, 4-chloro-2-(2-chloro-2-methylpropyl)-5-[(6-iodo-3-pyridinyl)methoxy]-3(2H)pyridazinone, 4-chloro-5-[(6-chloro-3-pyridinyl)methoxy]-2-(3,4-dichlorophenyl)-3(2H)pyridazinone, Bacillus thuringiensis strain EG-2348, [2-benzoyl-1-(1,1-dimethylethyl)-hydrazinobenzoic acid, 2,2-dimethyl-3-(2,4-dichlorophenyl)-2-oxo-1-oxaspiro[4.5]dec-3-en-4-yl butanoate, [3-[(6-chloro-3-pyridinyl)methyl]-2-thiazolidinylidene]-cyanamide, dihydro-2-(nitromethylene)-2H-1,3-thiazine-3 (4H)-carboxaldehyde, ethyl [2-[[1,6-dihydro-6-oxo-1-(phenylmethyl)-4-pyridazinyl]oxy]ethyl]-carbamate, N-(3,4,4-trifluoro-1-oxo-3-butenyl)-glycine, N-(4-chlorophenyl)-3-[4-(difluoromethoxy)phenyl]-4,5-dihydro-4-phenyl-1Hpyrazole-1-carboxamide, N-[(2-chloro-5-thiazolyl)methyl]-N′-methyl-N″-nitro-guanidine, N-methyl-N′-(1-methyl-2-propenyl)-1,2-hydrazinedicarbothioamide, N-methyl-N′-2-propenyl-1,2-hydrazinedicarbothioamide, O,O-diethyl [2-(dipropylamino)-2-oxoethyl]-ethylphosphoramidothioate, N-cyanomethyl-4-trifluoromethyl-nicotinamide, 3-5-dichloro-1-(3,3-dichloro-2propenyloxy)-4-[3-(5-trifluoromethylpyridin-2-yloxy)-propoxy]benzene. It is also possible to admix other known active compounds, such as herbicides, fertilizers and growth regulators.

When used as insecticides, the active compounds according to the invention can furthermore be present in their commercially available formulations and in the use forms, prepared from these formulations, as a mixture with synergistic agents. Synergistic agents are compounds which increase the action of the active compounds according to the invention, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as a good stability to alkali on limed substrates.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having specific properties (“traits”) which can be obtained by conventional breeding, by mutagenesis or by recombinant DNA techniques. This can be varieties, bio- and genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures, increased tolerance to drought.or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The preferred transgenic plants or plant cultivars (i.e. those obtained by genetic engineering) which are to be treated according to the invention include all plants which, in the genetic modification, received genetic material which imparted particularly advantageous useful properties (“traits,”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapevines), and particular emphasis is given to maize, soya beans, potatoes, cotton and oilseed rape. Traits that are emphasized are in particular increased defence of the plants against insects by toxins formed in the plants, in particular those formed by the genetic material from Bacillus thuringiensis (for example by the genes CryIA(a), CryIA(b), CryIA(c), CryIIA, CryIIIA, CryIIIB2,Cry9c, Cry2Ab, Cry3Bb and CryIF and also combinations thereof) (hereinbelow referred to as “Bt plants”). Traits that are also particularly emphasized are the increased defence of plants to fungi, bacteria and viruses by systemic acquired resistance (SAR), systemin, phytoalexius, elicitous and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS(® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these or still to be developed genetic traits, which plants will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or the mixtures specifically mentioned in the present text.

The active compounds according to the invention act not only against plant, hygiene and stored product pests, but also in the veterinary medicine sector against animal parasites (ectoparasites), such as hard ticks, soft ticks, mange mites, leaf mites, flies (biting and licking), parasitic fly larvae, lice, hair lice, feather lice and fleas. These parasites include: From the order of the Anoplurida, for example, Haematopinus spp., Linognathus spp., Pediculus spp., Phtirus spp. and Solenopotes spp.

From the order of the Mallophagida and the suborders Amblycerina and Ischnocerina, for example, Trimenopon spp., Menopon spp., Trinoton spp., Bovicola spp., Wemeckiella spp., Lepikentron spp., Damalina spp., Trichodectes spp. and Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopsylla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattella germanica and Supella spp.

From the subclass of the Acaria (Acarida) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

From the order of the Actinedida (Prostigmata) und Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp; Psoroptes spp., Chorioptes spp., Otodectes spp., Sarcoptes spp., Notoedres spp., Knemidocoptes spp., Cytodites spp. and Laminosioptes spp.

They have, for example, excellent activity against the development stages of ticks such as, for example, Amblyomma hebraeum and against parasitic flies such as, for example, Lucilia cuprina.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) according to the invention can be used as formulations (for example powders, emulsions, free-flowing compositions), which comprise the active compounds according to the invention in an amount of 1 to 80% by weight, directly or after 100 to 10 000-fold dilution, or they can be used as a chemical bath.

It has furthermore been found that the compounds according to the invention have a strong insecticidal action against insects which destroy industrial materials.

The following insects may be mentioned as examples and as preferred - but without a limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutus.

Hymenopterons, such as Sirex juvencus, Urocerus gigas, Urocerus gigas taignus, Urocerus augur.

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus.

Bristletails, such as Lepisma saccarina.

Industrial materials in the present connection are to be understood as meaning nonliving materials, such as, preferably, plastics, adhesives, sizes, papers and cards, leather, wood and processed wood products and coating compositions.

Wood and processed wood products are materials to be protected, especially preferably, from insect infestation.

Wood and processed wood products which can be protected by the agents according to the invention or mixtures comprising these are to be understood as meaning, for example:

building timber, wooden beams, railway sleepers, bridge components, boat jetties, wooden vehicles, boxes, pallets, containers, telegraph poles, wood panelling, wooden window frames and doors, plywood, chipboard, joinery or wooden products which are used-quite generally in house-building or in building joinery.

The active compounds according to the invention can be used as such, in the form of concentrates or in generally customary formulations, such as powders, granules, solutions, suspensions, emulsions or pastes.

The formulations mentioned can be prepared in a manner known per se, for example by mixing the active compounds according to the invention with at least one solvent or diluent, emulsifier, dispersing agent and/or binder or fixing agent, a water repellent, if appropriate siccatives and UV stabilizers and if appropriate dyestuffs and pigments, and also other processing auxiliaries.

The insecticidal compositions or concentrates used for the preservation of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by a series of tests. In general, however, it is sufficient to employ 0.0001 to 20% by weight, preferably 0.001 to 10% by weight, of the active compound, based on the material to be preserved.

Solvents and/or diluents which are used are an organic chemical solvent or solvent mixture and/or an oily or oil-like organic chemical solvent or solvent mixture of low volatility and/or a polar organic chemical solvent or solvent mixture and/or water, and if appropriate an emulsifier and/or wetting agent.

Organic chemical solvents which are preferably used are oily or oil-like solvents having an evaporation number above 35 and a flashpo,int above 30° C., preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkylbenzene.

Mineral oils having a boiling range from 170 to 220° C, white spirit having a boiling range from 170 to 220° C., spindle oil having a boiling range from 250 to 350° C., petroleum and aromatics having a boiling range from 160 to 280° C., terpentine oil and the like, are advantageously employed.

In a preferred embodiment, liquid aliphatic hydrocarbons having a boiling range from 180 to 210° C. or high-boiling mixtures of aromatic and aliphatic hydrocarbons having a boiling range from 180 to 220° C. and/or spindle oil and/or monochloronaphthalene, preferably a-monochloronaphthalene, are used.

The organic oily or oil-like solvents of low volatility which have an evaporation number above 35 and a flashpoint above 30° C., preferably above 45° C., can be replaced in part by organic chemical solvents of high or medium volatility, provided that the solvent mixture likewise has an evaporation number above 35 and a flashpoint above 30° C., preferably above 45° C., and that the insecticide/fungicide mixture is soluble or emulsifiable in this solvent mixture.

According to a preferred embodiment, some of the organic chemical solvent or solvent mixture is replaced by an aliphatic polar organic chemical solvent or solvent mixture. Aliphatic organic chemical solvents containing hydroxyl and/or ester and/or ether groups, such as, for example, glycol ethers, esters or the like, are preferably used.

Organic chemical binders which are used in the context of the present invention are the synthetic resins and/or binding drying oils which are known per se, are water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-coumarone resin, silicone resin, drying. vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odour correctants and inhibitors or anticorrosive agents and.the like which are known per se can additionally be employed.

It is preferred according to the invention for the composition or concentrate to comprise, as the organic chemical binder, at least one alkyd resin or modified alkyd resin and/or a drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phthalic acid esters, such as dibutyl, dioctyl or benzyl butyl phthalate, phosphoric acid esters, such as tributyl phosphate, adipic acid esters, such as di-(2-ethylhexyl) adipate, stearates, such as butyl stearate or amyl stearate, oleates, such as butyl oleate, glycerol ethers or higher molecular weight glycol ethers, glycerol esters and p-toluenesulphonic. acid esters.

Fixing agents are based chemically on polyvinyl alkyl ethers, such as, for example, polyvinyl methyl ether or ketones, such as benzophenone or ethylenebenzophenone.

Possible solvents or diluents are, in particular, also water, if appropriate as a mixture with one or more of the abovementioned organic chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Especially preferred mixing partners which may be mentioned are insecticides, such as chlorpyriphos, phoxim, silafluofm, alphamethrin, cyfluthrin, cypermethrin, deltamethrin, permethrin, imidacloprid, NI-25, flufenoxuron, hexaflumuron, transfluthrin, thiacloprid, methoxyfenozide and triflumuron, and also fungicides, such as epoxyconazole, hexaconazole, azaconazole, propiconazole, tebuconazole, cyproconazole, metconazole, imazalil, dichlorfluanid, tolylfluanid, 3-iodo-2propinyl-butyl carbamate, N-octyl-isothiazolin-3-one and 4,5-dichloro-N-octylisothiazolin-3-one.

The compounds according to the invention can at the same time be employed for protecting objects which come into contact with saltwater or brackish water, such as hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis(trialkyltin) sulphides, tri-n-butyltin laurate, tri-n-butyltin chloride, copper(I) oxide, triethyltin chloride, tri-n-butyl(2-phenyl-4-chlorophenoxy)tin, tributyltin oxide, molybdenum disulphide, antimony oxide, polymeric butyl titanate, phenyl(bispyridine)-bismuth chloride, tri-n-butyltin fluoride, manganese ethylenebisthio-carbamate, zinc dimethyldithiocarbamate, zinc ethylenebisthiocarbamate, zinc salts and copper salts of 2-pyridinethiol 1-oxide, bisdimethyldithiocarbamoylzinc ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combinations with the antifouling compositions according to the invention are:

algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;.

fungicides such as benzo[b]thiophenecarboxylic acid cyclohexylamide S,S-dioxide, dichlofluanid, fluorfolpet, 3-iodo-2-propinyl butylcarbamate, tolylfluanid and azoles such as azaconazole, cyproconazole, epoxyconazole, hexaconazole, metconazole, propiconazole and tebuconazole;

molluscicides such as fentin acetate, metaldehyde, methiocarb, niclosamid, thiodicarb and trimethacarb; or conventional antifouling active compounds such as 4,5-dichloro-2-octyl-4-isothiazolin-3-one, diiodomethylparatryl sulphone, 2-(N,Ndimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6tetrachloro-4-(methylsulphonyl)-pyridine, 2,4,5,6-tetrachloroisophthalonitrile, tetramethylthiuram disulphide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a. solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as colophonium to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds according to the invention are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all development stages. These pests include:

From the order of the Scorpionidea, for example, Buthus occitanus. From the order of the Acarina, for example, Argas persicus, Argas reflexus, Bryobia spp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae. From the order of the Araneae, for example, Aviculariidae, Araneidae. From the order of the Opiliones, for example, Pseudoscorpiones chelifer, Pseudoscorpiones cheiridium, Opiliones phalangium. From the order of the Isopoda, for example, Oniscus asellus, Porcellio scaber. From the order of the Diplopoda, for example, Blaniulus guttulatus, Polydesmus spp. From the order of the Chilopoda, for example, Geophilus spp. From the order of the Zygentoma, for example, Ctenolepisma spp., Lepisma saccharina, Lepismodes inquilinus. From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa. From the order of the Saltatoria, for example, Acheta domesticus. From the order of the Dermaptera, for example, Forficula auricularia. From the order of the Isoptera, for example, Kalotermes spp., Reticulitermes spp. From the order of the Psocoptera, for example, Lepinatus spp., Liposcelis spp. From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum. From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga camaria, Simulium spp., Stomoxys calcitrans, Tipula paludosa. From the order of the Lepidoptera, for example, Achroia grisella, Galleria mellonella, Plodia interpunctella, Tinea cloacella, Tinea pellionella, Tineola bisselliella. From the order of the Siphonaptera, for example, Ctenocephalides canis, Ctenocephalides felis, Pulex irritans, Tunga penetrans, Xenopsylla cheopis. From the order of the Hymenoptera, for example, Camponotus herculeanus, Lasius fuliginosus, Lasius niger, Lasius umbratus, Monomorium pharaonis, Paravespula spp., Tetramorium caespitum. From the order of the Anoplura, for example, Pediculus humanus capitis, Pediculus humanus corporis, Phthirus pubis. From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric acid esters, carbamates, pyrethroids, growth regulators or active compounds from other known classes of insecticides.

They are used as aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive evaporation systems, moth papers, moth bags and moth gels, as granules or dusts, in baits for spreading or in bait stations.

The preparation and use of the substances according to the invention is shown in the examples below.

### PREPARATION EXAMPLES

Process (A)

### Example 1

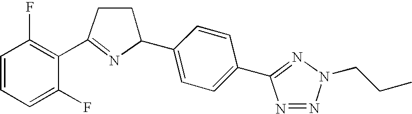

A mixture of 1 g (3 mmol) of 5-{4′-[5-(2,6-difluorophenyl)-3,4-dihydro-2H-pyrrol-2yl]-phenyl-4-yl}-2H-tetrazole (I-a-1), 0.44 g (3.6 mmol) of n-propyl bromide, 0.5 g (3.6 mmol) of potassium carbonate and 30 ml of acetonitrile is stirred at 80° C. for 18 hours. The solvent is then distilled off under reduced pressure, the residue is partitioned between ethyl acetate and water and the organic phase is separated off and dried over sodium sulphate. The solvent is distilled off, and the residue is then purified by silica gel chromatography (mobile phase: methylene chloride/diethyl ether=7:1).

hThis gives 0.19 g (17%) of 5-{4′-[5-(2,6-difluorophenyl)-3,4-dihydro-2H-pyrrol-2yl]-l,1′-biphenyl-4-yl}-2-propyl-2H-tetrazole in the form of a viscous oil. HPLC: logP (pH 2.3)=2.61

Preparation of Starting Materials of the Formula (I-a)

### Example (I-a-1)

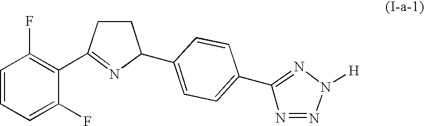

A mixture of 1 g (3.5 mmol) of 4-[5-(2,6-difluorophenyl)-3,4-dihydro-2H-pyrrol-2yl]benzonitrile (VIII-1), 1 g (4.8 mmol) of trimethyltin azide and 30 ml of toluene is boiled under reflux for 18 hours. The solvent is then distilled off under reduced pressure and the residue is dissolved in 25 ml of 5 percent strength aqueous sodium hydroxide solution. Undissolved components are removed by filtration. The filtrate is then adjusted to about pH 5 by addition of dilute hydrochloric acid. The mixture is then extracted with ethyl acetate and the organic phase is separated off and dried over sodium sulphate. According to HPLC analysis, the resulting residue (1.5 g) comprises 60% 5-{4′-[5-(2,6-difluorophenyl)-3,4-dihydro-2H-pyrrol-2-yl]-phenyl-4yl}-2H-tetrazole (yield: 79%)

HPLC: logP (pH 2.3)=1.28

Preparation of Starting Materials of the Formula (VIII)

### Example (VIII-1)

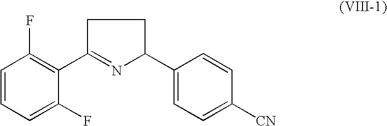

Trifluoroacetic acid (2.85 g, 25.0 mmol) is initially charged at 5° C. A solution of N-[ 1-(4-cyanophenyl)-4-(2,6-difluorophenyl)-4-oxobutyl]acetamide (X-1) (0.50 g, 75%, 0.94 mmol) in dichloromethane (10 ml) is slowly added dropwise. The mixture is stirred at room temperature for 3 hours and then evaporated to dryness. The residue is taken up in ethyl acetate (50 ml) and washed with 1N aqueous sodium hydroxide soludion (50 ml). The organic phase is washed with water, dried over magnesium sulphate, filtered and concentrated. The crude product is purified by silica gel chromatography (mobile phase: cyclohexane/ethyl acetate 7:3).

This gives 0.19 g (69% of theory) of 4-[5-(2,6-difluorophenyl)-3,4-dihydro-2Hpyrrol-2-yl]benzonitrile. HPLC: logP (pH 2.3)=2.11 (96.6%) M.p.: 57° C. NMR (CD₃CN): δ=1.77 (1H, m), 2.65 (1H, m), 3.06 (2H, m), 5.35 (1H, m), 7.09 (2H, m), 7.48 (3H, m), 7.72 (2H, d) ppm.

### Example (VIII-2)

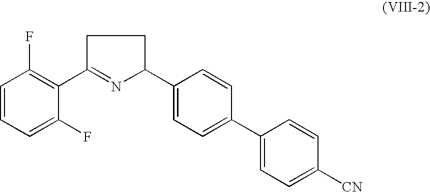

Analogously to Example (VIII-1), reaction of N-[1-(4′-cyano-1,1′-biphenyl-4-yl)-4(2,6-difluorophenyl)-4-oxobutyl]acetamide (X-2) with trifluoracetic acid gives the compound 4′-[5-(2,6-difluorophenyl)-3,4-dihydro-2H-pyrrol-2-yl]-1,1′-biphenyl-4carbonitrile.

HPLC: logP (pH 2.3)=2.84 M.p.: 123° C. NMR (CD₃CN): δ=1.83 (1H, m), 2.64 (1H, m), 3.07 (2H, m), 5.35 (1H, m),7.09 (2H, m), 7.48 (3H, m), 7.69 (2H, d), 7.81 (4H, s) ppm.

Preparation of Starting Materials of the Formula (X)

### Example (X-1)

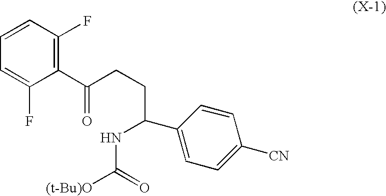

1,3-Difluorobenzene (5.07 g, 44.45 mmol) is initially charged in tetrahydrofuran (100 ml) and cooled to −78° C. At this temperature, n-butyllithium (28.25 ml, 1.6 M in hexane, 44.45 mmol) is added dropwise, and the mixture is stirred at -78° C. for another half an hour. A solution of tert-butyl-2-(4-cyanophenyl)-5-oxo-1-pyrrolidinecarboxylate (XI-1) (11.57 g, 40.41 mmol) in tetrahydrofuran (50 ml) is added dropwise at −78° C., and the reaction mixture is stirred at this temperature for another 3 hours. The mixture is carefully stirred into water (100 ml) and extracted with ethyl acetate (2×150 ml). The combined organic phases are dried over magnesium sulphate, filtered and concentrated. The crude product is purified by silica gel chromatography (mobile phase: cyclohexane/ethyl acetate 20:1→10:1).

This gives 16.55 g (76% of theory) of N-[1-(4-cyanophenyl)-4-(2,6-difluorophenyl)-4-oxobutyl]acetamide. HPLC: logP (pH 2.3)=3.36 (74.57%)

### Example (X-2)

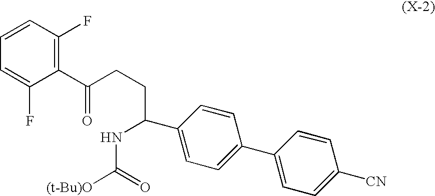

Analogously to Example (X-1), reaction of tert-butyl-2-(4′-cyano-1,1′-biphenyl-4-yl)-5-oxo-1-pyrrolidinecarboxylate (XI-2) with 1,3-difluorobenzene und n-butyllithium gives the compound N-[1-(4′-cyano-1,1′-biphenyl-4-yl)-4-(2,6-difluorophenyl)-4oxobutyl]acetamide. HPLC: logP (pH2.3)=4.10

Preparation of Starting Materials of the Formula (XI)

### Example (XI-1)

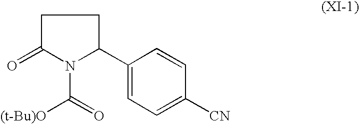

4-(5-Oxo-2-pyrrolidinyl)benzonitrile (XIII-a-1) (9.99 g, 48.33 mmol) is initially charged in dichloromethane (90 ml). At 0-5° C., a solution of di-tert-butyl carbonate (21.09 g, 96.66 mmol) in dichloromethane (40 ml), dimethylaminopyridine (6.02 g, 49.30 mmol) and triethylamine (4.99 g, 49.30 mmol) are slowly added in succession (dropwise, if appropriate). The mixture is stirred at room temperature for 60 hours and washed successively with citric acid (20 ml, 10% w/v) and saturated sodium bicarbonate solution (20 ml). The organic phase is dried over magnesium sulphate, filtered and concentrated under reduced pressure. The crude product is purified by silica gel chromatography (mobile phase: toluene/ethyl acetate 4:1).

This gives 8.01 g (58% of theory) of tert-butyl-2-(4-cyanophenyl)-5-oxo-1-pyrrolidinecarboxylate. HPLC: logp (pH 2.3)=2.12 (100%) NMR(CD₃CN): δ=1.76 (1H, m), 2.44-2.55 (3H, m), 5.17 (1H, m), 7.42 (2H, d), 7.72 (2H, m) ppm.

### Example (XI-2)

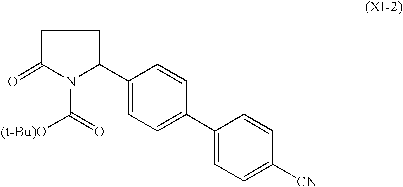

Analogously to Example (XI-1), reaction of 4′-(5-oxo-2-pyrrolidinyl)-1,1′-biphenyl-4-carbonitrile (XIII-b-1) with di-tert-butyl carbonate gives the compound tert-butyl 2(4′-cyano-1,1′-biphenyl-4-yl)-5-oxo-1-pyrrolidinecarboxylate. HPLC: logP (pH 2.3) 3.03 M.p.: 152° C.

Preparation of Starting Materials of the Formula (XIII)

### Example (XIII-a-1)

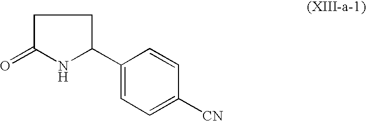

4-(5-Oxo-2-pyrrolidinyl)phenyl-trifluoromethanesulphonate (6.19 g, 20 mmol), zinc(II) cyanide (1.65 g, 14.0 mmol) and tetrakis(triphenylphosphine)palladium (0.92 g, 0.79 mmol) are stirred in dimethylformamide (30 ml) at 80° C. for 45 minutes. The mixture is cooled to room temperature, poured into saturated aqueous sodium bicarbonate solution (50 ml) and extracted with ethyl acetate (3×100 ml). The combined organic phases are dried over magnesium sulphate, filtered and concentrated under reduced pressure. This gives 2.27 g (60% of theory) of 4-(5-oxo-2-pyrrolidinyl)benzonitrile.

HPLC: logP (pH 2.3)=0.96 (98.42%) M.p.: 155° C. NMR (CD₃CN): δ=1.82 (1H, m), 2.29 (2H, m), 2.56 (1H, m), 4.78 (1H, m), 6.45 (1H, br), 7.49 (2H, d), 7.72 (2H, m) ppm.

### Example (XIII-b-1)

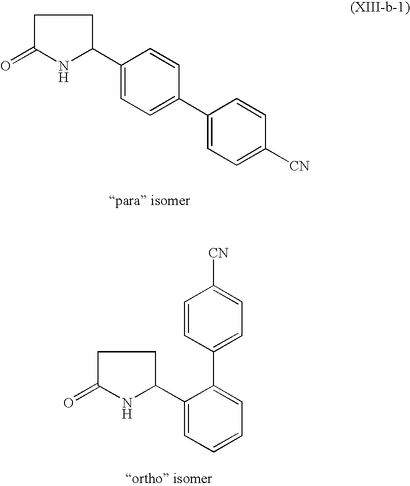

Hydrogen fluoride (50 ml) is initially charged at 0° C. A solution of 5-ethoxy-2pyrrolidinone (2.58 g, 0.02 mol) and 1,1′-biphenyl-4-caibonitrile (1.79 g, 0.01 mol) in dichloromethane (15 ml) is added dropwise. The reaction mixture is then stirred at room temperature. HF is removed under reduced pressure and the residue is taken up in dichloromethane and washed with saturated aqueous sodium bicarbonate solution. The organic phase is dried over sodium sulphate, filtered and concentrated. The crude product is purified by silica gel chromatography (mobile phase: dichloromethane/ethyl acetate 1: 1).

This gives 0.74 g (27% of theory) of 4′-(5-oxo-2-pyrrolidinyl)-1,1′-biphenyl-4carbonitrile (“para” isomer). HPLC: logP (pH 2.3)=1.92 (94.4% purity) NMR (CDCl₃): δ=2.05 (1H, m), 2.46-2.63 (3H, m), 4.83 (1H, m), 5.86 (1H, br), 7.42 (2H, d), 7.61 (2H, d), 7.68 (2H, d), 7.74 (2H, d) ppm.

This gives 0.20 g (7% of theory) of 2′-(5-oxo-2-pyrrolidinyl)-1,1′-biphenyl-4carbonitrile (“ortho” isomer). HPLC: logP (pH 2.3)=1.94 (94.4% purity).

Process (B)

### Example 2

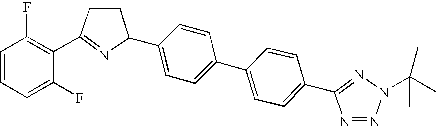

At room temperature, 1.1 g (4.5 mmol) of 4-[(2-tert-butyl)-2H-tetrazol-5-yl]phenylboronic acid (V-1) and 185 mg of tetrakis-(triphenylphosphine)-palladium(0) are added to a solution of 1 g (4.3 mmol) of 2-(2,6-difluorophenyl)-5-(4bromophenyl)-3,4-dihydro-2H-pyrrole in 10 ml of 1,2-dimethoxyethane, and the mixture is stirred at room temperature for 15 minutes. 4.8 ml of sodium carbonate solution (2 M in water) are then added, and the reaction mixture is stirred at 80° C. for 16 hours. After cooling to room temperature, the reaction mixture is poured into about 100 ml of water and extracted twice with ethyl acetate. The organic phases are washed with concentrated sodium chloride solution, dried over sodium sulphate and concentrated under reduced pressure. The residue is purified by silica gel chromatography (mobile phase: methylene chloride/ether=5:1).

This gives 0.51 g (37% of theory) of 2-tert-butyl-5-{4′-[5-(2,6-difluorophenyl)-3,4dihydro-2H-pyrrol-2-yl]-1,1′-biphenyl-4-yl}-2H-tetrazole in the form of a viscous oil which slowly crystallizes. HPLC: logP (pH 2.3)=4.14 (94%) M.p. 96° C.

Preparation of Starting Materials of the Formula (V)

### Example (V-1)

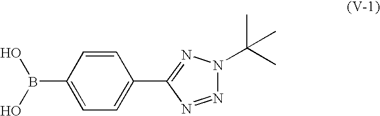

At −78° C., 27.8 ml of a 2.5M solution of butyllithium in hexane (0.072 mol) are added dropwise to a solution of 18.4 g (0.066 mol) of 2-tert-butyl-5-(4-bromophenyl)-1 (2)H-tetrazole in 150 ml of tetrahydrofuran. The mixture is stirred at −78° C. for 15 minutes, and 8 g (0.077 mol) of trimethyl borate are then added dropwise at the same temperature. The temperature of the mixture is then allowed to slowly warm to room temperature, and the mixture is stirred at room temperature for another 18 hours. After addition of 50 ml of water, the solvent is removed under reduced pressure and the residue is dissolved in dilute aqueous sodium hydroxide solution. The solution is filtered through Celite, the filtrate is acidified with dilute hydrochloric acid and the precipitated product obtained after crystallization is filtered off with suction.

This gives 11.2 g (74% of theory) of 4-[(2-tert-butyl)-2H-tetrazol-5-yl]phenylboronic acid as a slightly yellowish powder. HPLC: logP (pH 2.3)=2.01

### Example (V-2)

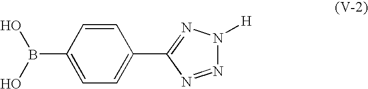

Analogously to Example (V-1), reaction of 5-(4-bromo-phenyl)-2H-tetrazole gives the compound ⁴-(2H-tetrazol-5-yl)-phenylboronic acid. HPLC: logp (pH 2.3)=0.19

Process (D)

### Example 3

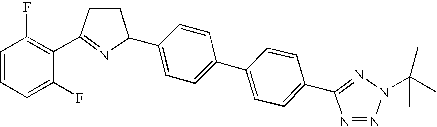

Under argon, 4-[5-(2,6-difluorophenyl)-3,4-dihydro-2H-pyrrol-2-yl]phenyltrifluoromethanesulphonate (10.13 g, 25.0 mmol), bis(pinacolato)diboron (7.62 g, 30.0 mmol), potassium acetate (7.36 g, 75.0 mmol) and PdCl₂dppf (0.56 g, 0.77 mmol) are heated in dimethylacetamide (150 ml) at 80° C. for 3 hours. After cooling, ²-tert-butyl-5-(4-bromo-phenyl)-1(2)H-tetrazole (VII-1) (7.73 g, 27.50 mmol), PdCl₂dppf (0.56 g, 0.77 mmol) and sodium carbonate solution (75 ml, 2M in water) are added, and the mixture is stirred at 80° C. for 16 hours. The reaction mixture is poured into water (600 ml) and extracted with ethyl acetate (2×500 ml). The combined organic phases are dried over sodium sulphate and filtered. Florisil (60 g) is added, and the solvent is removed under reduced pressure. The crude product (which is absorbed on Florisil) is purified by silica gel chromatography (mobile phase: n-hexane/ethyl acetate 4:1→3:1).

This gives 6.30 g (54% of theory) of 2-tert-butyl-5-{4′-[5-(2,6-difluorophenyl)-3,4dihydro-2H-pyrrol-2-yl]-1′-biphenyl-4-yl }-2H-tetrazole. HPLC: logP=4.14 (98.9%)

### Example 4

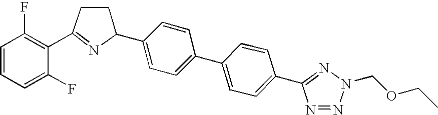

Analogously to Example 3,4-[5-(2,6-difluorophenyl)-3,4-dihydro-2H-pyrrol-2yl]phenyl-trifluoromethanesulphonate and 2-ethoxymethyl-5-(4-bromo-phenyl)-1(2)H-tetrazole (VII-2) are reacted to give 5-{4′-[5-(2,6-difluorophenyl)-3,4-dihydro-2H-pyrrol-2-yl]-1,1′-biphenyl-4-yl}-2-(ethoxymethyl)-2H-tetrazole.

HPLC: logP=3.40 (98.99%) NMR(CD₃CN): δ=1.17 (3H, t), 1.85 (1H, m), 2.64 (1H, m), 3.07 (2H, m), 3.70 (2H, q), 5.36 (1H, m), 5.94 (2H? s), 7.09 (2H, m), 7.47 (3H, m), 7.72 (2H, d), 7.84 (2H, d), 8.22 (2H, d) ppm.

### Example 5

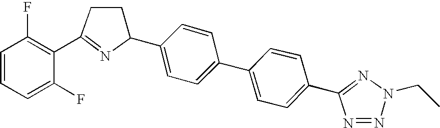

Under argon, 2-(4-bromophenyl)-5-(2,6-difluorophenyl)-3,4-dihydro-2H-pyrrole (0.84 g, 2.50 mmol), bis(pinacolato)diboron (0.76 g, 3.0 mmol), potassium acetate (0.74 g, 7.50 mmol) and PdCl₂dppf (60 mg, 0.075 mmol) are heated in dimethylfonnamide (15 ml) are heated at 80° C. for 2 hours. After cooling, 2-ethyl-5(4-bromo-phenyl)-1(2)H-tetrazole (VII-3) (0.76 g, 3.0 mmol), PdCl₂dppf (60 mg, 0.075 mmol) and sodium carbonate solution (7.5 ml, 2M in water) are added, and the mixture is stirred at 80° C. for 16 hours. The reaction mixture is poured into water (60 ml) and extracted with ethyl acetate (2×50 ml). The combined organic phases are dried over sodium sulphate and filtered. Florisil (6.0 g) is added, and the solvent is removed under reduced pressure. The crude product (which is absorbed on Florisil) is purified by silica gel chromatography (mobile phase: n-hexane/ethyl acetate 9:1→3:1).

This gives 0.66 g (62% of theory) of 5-{4′-[5-(2,6-difluorophenyl)-3,4-dihydro-2Hpyrrol-2-yl]-1,1′-biphenyl-4-yl}-2-ethyl-2H-tetrazole. HPLC: logP (pH 2.3)=3.21 (91.98%) NMR (CD₃CN): δ=1.64 (3H, t), 1.85 (1H, m), 2.65 (1H, m), 3.08 (2H, m), 4.71 (2H, q), 5.34 (1H, m), 7.09 (2H, m), 7.47 (3H, m), 7.72 (2H, d), 7.82 (2H, d), 8.18 (2H, d) ppm.

Preparation of Starting Materials of the Formula (VII)

### Example (VII-1)

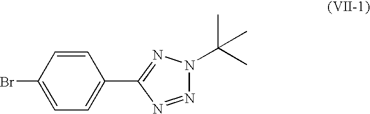

A mixture of 20 g (0.089 mol) of 5-(4-bromo-phenyl)-1(2)H-tetrazole, 20 ml of tertbutanol, 5.4 ml of concentrated sulphuric acid and 100 ml of trifluoroacetic acid is stirred at room temperature for 18 hours. The trifluoroacetic acid is then distilled off under reduced pressure, the residue is partitioned between ethyl acetate and concentrated sodium bicarbonate solution and the organic phase is separated off and dried over sodium sulphate. The solvent is then removed under reduced pressure.

This gives 18.4 g (74% of theory) of 2-tert-butyl-5-(4-bromo-phenyl)-1(2)H-tetrazole. HPLC: logP (pH 2.3)=4.23

### Example (VII-2)

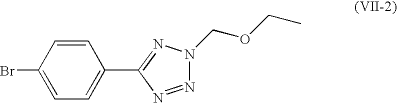

A mixture of 5.6 g (0.025 mol) of 5-(4-bromo-phenyl)-1(2)H-tetrazole, 2.8 g (0.03 mol) of chloromethyl ethyl ether, 4.1 g (0.03 mol) of potassium carbonate and 150 ml of acetonitrile is stirred at 60° C. for 18 hours. The solvent is then distilled off under reduced pressure, the residue is partitioned between ethyl acetate and water and the organic phase is separated off and dried over sodium sulphate. The solvent is distilled off, and the residue is then purified by silica gel chromatography (mobile phase: methylene chloride).

This gives 3 g (43% of theory) of 2-ethoxymethyl-5-(4-bromo-phcnyl)-1(2)H tetrazole as a colourless powder. HPLC: logP (pH2.3)=3.42

### Example (VII-3)

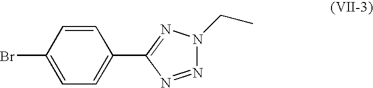

Analogously to Example (VII-2), reaction of 5-(4-bromo-phenyl)-1(2)H-tetrazole with ethyl iodide gives the compound 2-ethyl-5-(4-bromo-phenyl)-1(2)H-tetrazole.

HPLC: logP (pH 2.3)=3.20

The stated logP values were determined in accordance with EEC Directive 79/831 Annex V.A8 by HPLC (High Performance Liquid Chromatography) using a reversed-phase column (C 18). Temperature: 43° C.

In the acidic range, the determination was carried out at pH 2.3 using the mobile phases 0.1% aqueous phosphoric acid and acetonitrile; linear gradient from 10% acetonitrile to 90% acetonitrile.

In the neutral range, the determination was carried out at pH 7.5 using the mobile phases 0.01 molar aqueous phosphate buffer solution and acetonitrile; linear gradient from 10% acetonitrile to 90% acetonitrile.

Calibration was carried out using unbranched alkan-2-ones (having 3 to 16 carbon atoms) with known logP values (determination of the logP values by the retention times using linear interpolation between two successive alkanones).

The lambda max values were determined in the maxima of the chromatographic signals using the UV spectra from 200 nm to 400 nm.

### USE EXAMPLES

### Example A

Heliothis virescens Test

Solvent: 30 parts by weight of dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Soyabean shoots (Glycine max) are treated by being dipped into the preparation of active compound of the desired concentration and are populated with Heliothis virescens caterpillars whilst the leaves are still moist.

After the desired period of time, the kill in % is determined. 100% means that all caterpillars have been killed; 0% means that none of the caterpillars have been killed.

Active compounds, active compound concentrations and test results are shown in the table below.

TABLE A
Plant-damaging insects
Heliothis virescens test

| | Active | |
|---|---|---|
| | compound | |
| | concentration | Kill rate |
| Active compounds | in ppm | in % after 6^{d} |
| 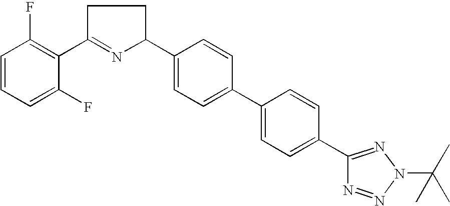 | 40 | 100 |
| 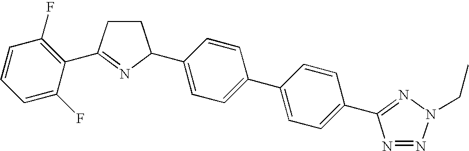 | 40 | 100 |

### Example B

Phaedon larvae Test

Solvent: 30 parts by weight of dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with larvae of the mustard beetle (Phaedon cochleariae) whilst the leaves are still moist.

After the desired period of time, the kill in % is determined. 100% means that all beetle larvae have been killed; 0% means that none of the beetle larvae have been killed.

Active compounds, active compound concentrations and test results are shown in the table below.

TABLE B
Plant-damaging insects
Phaedon larvae test

| | Active | |
|---|---|---|
| | compound | |
| | concentration | Kill rate |
| Active compounds | in ppm | in % after 7^{d} |
| 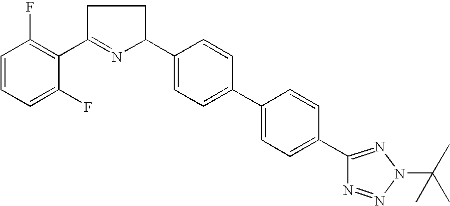 | 1000 | 100 |
| 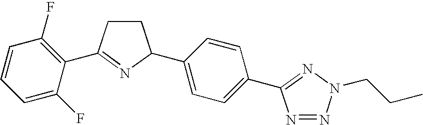 | 1000 | 100 |
| 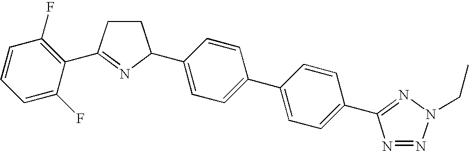 | 1000 | 100 |
| 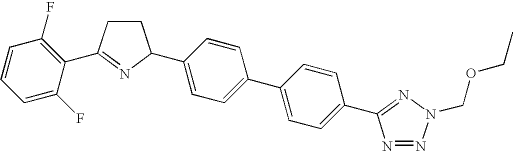 | 1000 | 100 |

### Example C

Plutella Test

Solvent: 30 parts by weight of dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the diamond-back moth (Plutella xylostella) whilst the leaves are still moist.

After the desired period of time, the kill in % is determined. 100% means that all caterpillars have been killed; 0% means that none of the caterpillars have been killed.

Active compounds, active compound concentrations and test results are shown in the table below.

TABLE C
Plant-damaging insects
Plutella test

| | Active | |
|---|---|---|
| | compound | |
| | concentration | Kill rate |
| Active compounds | in ppm | in % after 6^{d} |
| 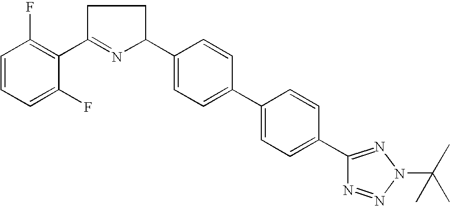 | 40 | 100 |

### Example D

Spodoptera exigua Test

Solvent: 7 parts by weight of dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration. Cabbage leaves (Brassica oleracea) are treated by being dipped into the preparation of active compound of the desired concentration and are populated with army worm (Spodoptera exigua) caterpillars whilst the leaves are still moist.

After the desired period of time, the kill in % is determined. 100% means that all caterpillars have been killed; 0% means that none of the caterpillars have been killed.

Active compounds, active compound concentrations and test results are shown in the table below.

TABLE D
Plant-damaging insects
Spodoptera exigus test

| | Active | |
|---|---|---|
| | compound | |
| | concentration | Kill rate |
| Active compounds | in ppm | in % after 6^{d} |
| 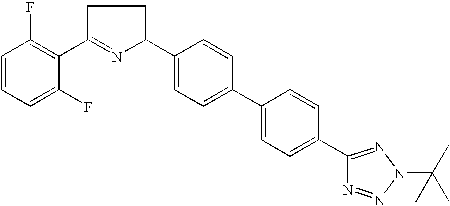 | 8 | 100 |
| 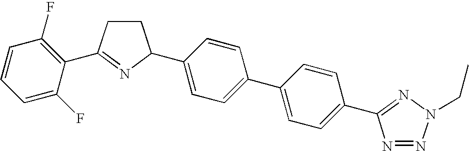 | 40 | 100 |

### Example E

Spodoptera frugiperda Test

Solvent: 30 parts by weight of dimethylformamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with army worm (Spodoptera frugiperda) caterpillars whilst the leaves are still moist.

After the desired period of time, the kill in % is determined. 100% means that all caterpillars have been killed; 0% means that none of the caterpillars have been killed.

Active compounds, active compound concentrations and test results are shown in the table below.

TABLE E
Plant-damaging insects
Spodoptera frugiperda test

| | Active | |
|---|---|---|
| | compound | |
| | concentration | Kill rate |
| Active compounds | in ppm | in % after 7^{d} |
| 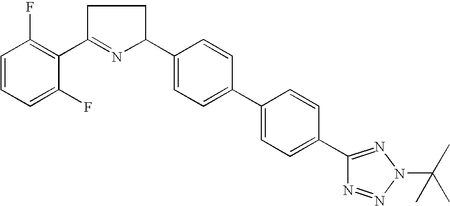 | 1000 | 100 |
| 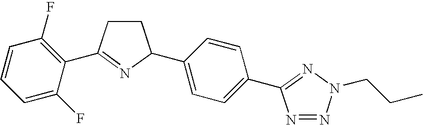 | 1000 | 100 |
| 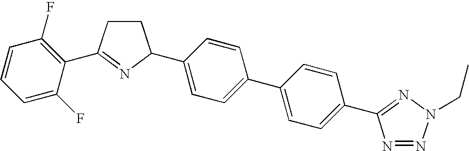 | 1000 | 100 |
| 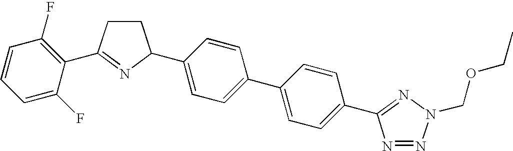 | 1000 | 100 |

### Example F

Tetranychus Test (OP-Resistant/Dip Treatment)

Solvent: 30 parts by weight of dimethylformamide

Emulsifier: 1 part by weight of alkylaryl polyglycol ether

To produce a suitable preparation of active compound, 1 part by weight of active compound is mixed with the stated amounts of solvent and emulsifier, and the. concentrate is diluted with emulsifier-containing water to the. desired concentration.

Bean plants (Phaseolus vulgaris) which are heavily infested by all stages of the greenhouse red spider mite (tetranychus urticae) are dipped into a preparation of active compound of the desired concentration.

After the desired period of time, the effect in % is determined. 100% means that all spider mites have been killed; 0% means that none of the spider mites have been killed.

Active compounds, active compound concentrations and test results are shown in the table below.

TABLE F
Plant-damaging mites
Tetranychus test (OP-resistant/dip treatment)

| | Active | |
|---|---|---|
| | compound | |
| | concentration | Kill rate |
| Active compounds | in ppm | in % after 7^{d} |
| 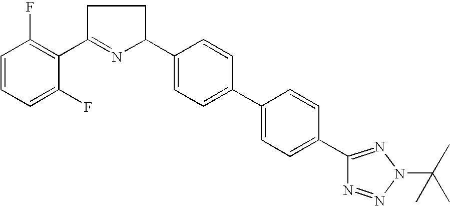 | 100 | 98 |
| 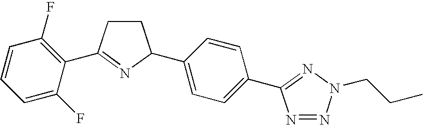 | 100 | 90 |
| 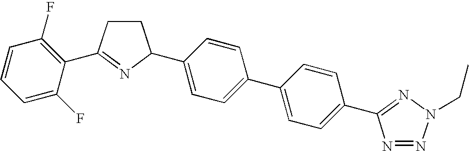 | 100 | 98 |
| 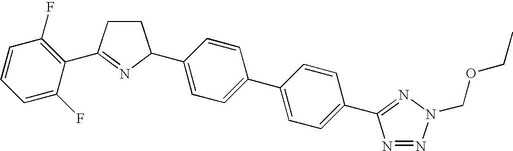 | 100 | 98 |

### Example G

Blowfly Larvae Test/Development-Inhibitory Action

Test animals: Lucilia cuprina larvae

Solvent: Dimethyl sulphoxide

20 mg of active compound are dissolved in 1 ml of dimethyl sulphoxide, more dilute concentrations are prepared by dilution with distilled water.

About 20 Lucilia cuprina larvae are introduced into a test tube which contains about 1 cm³ of horse meat and 0.5 ml of the preparation of active compound to be tested. The efficacy of the preparation of active compound is determined after 24 and 48 hours. The test tubes are transferred into beakers whose bottom is covered with sand. After a further 2 days, the test tubes are removed and the pupae are counted.

The activity of the preparation of active compound is assessed by the number of flies that have hatched after 1.5 times the development time of an untreated control. 100% means that no flies have hatched; 0% means that all flies have hatched normally.

Active compounds, active compound concentrations and test results are shown in the table below.

TABLE G
Development-inhibitory action
Blowfly larvae test

| | Active | |
|---|---|---|
| | compound | |
| | concentration | Kill rate |
| Active compounds | in ppm | in % after 48^{h} |
| 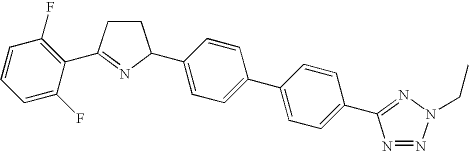 | 100 | 100 |
| 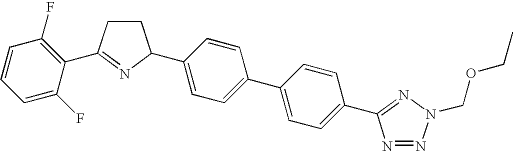 | 100 | 70 |

### Example H

Nymphecdysis Test on Polyphagous Ticks

Test animals: Amblyomma variegatum or A. hebraeum, nymphs which have sucked themselves full

Solvent: Dimethyl sulphoxide

20 mg of active compound are dissolved in 1 ml of dimethyl sulphoxide, more dilute concentrations are prepared by dilution with distilled water.

10 nymphs which have sucked themselves full are immersed for 1 minute into the preparation of active compound to be tested. The animals are transferred to petri dishes (∅ 9.5 cm) fitted with filter discs and covered. After the nymphs have remained in a controlled-environment cabinet for 4 to 6 weeks, the effect on ecdysis is determined.

100% means that none of the animals have undergone normal ecdysis. 0% means that all animals have undergone ecdysis.

In this test, for example, the compounds 4 and 5 of the preparation examples show good activity.

TABLE H
Nymphedysis test on polyphagous ticks

| | Active | |
|---|---|---|
| | compound | Effect |
| | concentration | on ecdysis |
| Active compounds | in ppm | in % after 48^{h} |
| 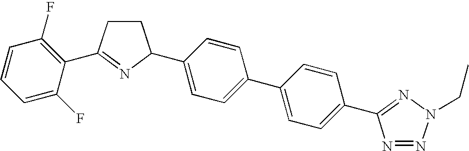 | 100 | 100 |
| 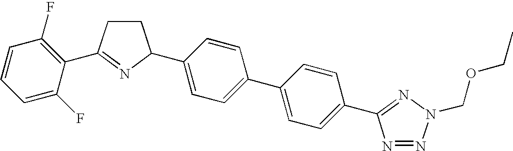 | 100 | 100 |

### Example J

Diabrotica balteata Test (Larvae in the Soil)

Critical concentration test/soil insects—treatment of transgenic plants

Solvent: 7 parts by weight of dimethylformamide

Emulsifier: 1 part by weight of alkylaryl polyglycol ether

To produce a suitable preparation of active compound, 1 part by weight of active compound is mixed with the stated amount of solvent, the stated amount of emulsifier is added and the concentrate is diluted with water to the desired concentration.

The preparation of active compound is poured onto the soil. Here, the concentration of the active compound in the preparation is virtually immaterial, only the amount by weight of active compound per volume unit of soil, which is stated in ppm (mg/l), matters. The soil is filled into 0.25 1 pots, and these are allowed to stand at 20° C.

Immediately after the preparation, 5 pregerminated maize corns of the cultivar YIELD GUARD (trade mark of Monsanto Comp., USA) are placed into each pot. After 2 days, the corresponding test insects are placed into the treated soil. After a further 7 days, the efficacy of the active compound is determined by counting the number of maize plants that have emerged (1 plant=20% activity).

### Example K

Heliothis virescens Test (Treatment of Transgenic Plants)

Solvent: 7 parts by weight of dimethylformamide

Emulsifier: 1 part by weight of alkylaryl polyglycol ether

To produce a suitable preparation of active compound, 1 part by weight of active compound is mixed with the stated amount of solvent and the stated amount of emulsifier, and the concentrate is diluted with water to the desired concentration. Soyabean shoots (Glycine max) of the cultivar Roundup Ready (trade mark of Monsanto Comp. USA) are treated by being dipped info the preparation of active compound of the desired concentration and populated with the tobacco buttworm caterpillar Heliothis virescens whilst the leaves are still moist.

After the desired period of time, the kill in % is determined. 100% means that all caterpillars have been killed; 0% means that none of the caterpillars have been killed.

1. Δ¹-Pyrrolines of the formula (I) 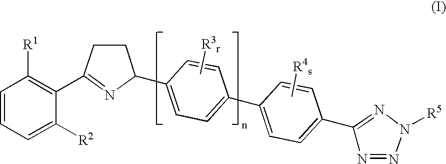
in which
R² represents halogen or methyl,
R² represents hydrogen or halogen,
R³ and R⁴ independently of one another represent halogen or represent in each case optionally substituted alkyl or alkoxy,
R⁵ represents hydrogen, alkylcarbonyl or represents in each case optionally substituted alkyl, alkylsulphonyl or 1-methyl-cycloalkyl,
n represents 0 or 1,
r and s independently of one another represent 0, 1 or 2.

2. Δ¹-Pyrrolines of the formula (I) according to claim 1, in which
R¹ represents halogen or methyl,
R² represents hydrogen or halogen,
R³ and R⁴ independently of one another represent halogen, alkyl, halogenoalkyl, alkoxy or halogenoalkoxy,
R⁵ represents hydrogen, alkylcarbonyl, alkyl, halogenoalkyl, alkoxyalkyl, alkoxycarbonylalkyl, alkylsulphonyl, halogenoalkylsulphonyl or 1-methyl-cycloalkyl, which may optionally be mono- to trisubstituted by alkyl,
n represents 0 or 1,
r and s independently of one another represent 0, 1 or 2.

3. Δ¹-Pyrrolines of the formula (I) according to claim 1, in which
R¹ represents fluorine, chlorine or methyl,
R² represents hydrogen, fluorine or chlorine,
R³ and R⁴ independently of one another represent fluorine, chlorine, bromine, C₁-C₆-alkyl, C₁-C₆-halogenoalkyl, C₁-C₆-alkoxy or C₁-C₆-halogenoalkoxy,
R⁵ represents hydrogen, C₁-C₆-alkylcarbonyl, C₁-C₈-alkyl, C₁-C₆-halogenoalkyl, C₁-C₆-alkoxy-C₁-C₆-alkyl, C₁-C₆-alkoxycarbonyl-C₁-C₆-alkyl, C₁-C₆-alkylsulphonyl, C₁-C₆-halogenoalkylsulphonyl or 1-methyl-C₃-C₆-cycloalkyl, which may optionally be mono- to trisubstituted by C₁-C₄-alkyl,
n represents 0 or 1,
r and s independently of one another represent 0, 1 or 2.

4. Δ¹-Pyrrolines of the formula (I) according to claim 1, in which
R¹ represents fluorine, chlorine or methyl,
R² represents hydrogen, fluorine or chlorine,
R³ and R⁴ independently of one another represent fluorine, chlorine, C₁-C₄alkyl, C₁-C₄-halogenoalkyl having 1 to 9 fluorine, chlorine and/or bromine atoms, C₁-C₄-alkoxy or C₁-C₄-halogenoalkoxy having 1 to 9 fluorine, chlorine and/or bromine atoms,
R⁵ represents hydrogen, C₁-C₄-alkylcarbonyl, C₁-C₈-alkyl, C₁-C₆-halogenoalkyl having 1 to 13 fluorine, chlorine and/or bromine atoms, C₁-C₄-alkoxy-C₁-C₄-alkyl, C₁-C₄-alkoxycarbonyl-C₁-C₄-alkyl, C₁-C₄-alkylsulphonyl, C₁-C₄-halogenoalkylsulphonyl having 1 to 9 fluorine, chlorine and/or bromine atoms, 1-methyl-C₃-C₆-cycloalkyl,
n represents 0 or 1,
r and s independently of one another represent 0, 1 or 2.

5. Δ¹-Pyrrolines of the formula (I) according to claim 1, in which
R¹ represents fluorine or chlorine,
R² represents hydrogen or fluorine,
R³ and R⁴ independently of one another represent fluorine, chlorine, methyl, ethyl, n-propyl, isopropyl, n-butyl, isobutyl, sec-butyl, tert-butyl, trifluoromethyl, trifluoroethyl, methoxy, ethoxy, n-propoxy, isopropoxy, n-butoxy, isobutoxy, sec-butoxy, tert-butoxy, trifluoromethoxy or trifluoroethoxy,
R⁵ represents hydrogen, methylcarbonyl, ethylcarbonyl, n-propylcarbonyl, isopropylcarbonyl, n-butylcarbonyl, isobutylcarbonyl, sec-butylcarbonyl, tert-butylcarbonyl, methyl, ethyl, n-propyl, isopropyl, n-butyl, isobutyl, sec-butyl, tert-butyl, pentyl, hexyl, heptyl, octyl, difluoromethyl, trifluoromethyl, trifluoroethyl, nonafluorobutyl, —CH₂OMe, —CH₂OEt, —CH₂O(t-Bu), —CH₂CO₂Me, —CH₂CO₂Et, —CH₂CO₂(n-Pr), —CH₂CO₂(i-Pr), —CH₂CO₂(s-Bu), —CH₂CO₂(i-Bu), —CH₂CO₂(t-Bu), —SO₂Me, —SO₂Et, —SO₂(n-Pr), —SO₂(i-Pr), —SO₂(t-Bu), —SO₂CF₃, —SO₂(CF₂)₃CF₃ or 1-methyl-cyclohexyl,
n represents 0 or 1,
r and s independently of one another represent 0, 1 or 2.

6. Δ¹-Pyrrolines of the formula (I-b) 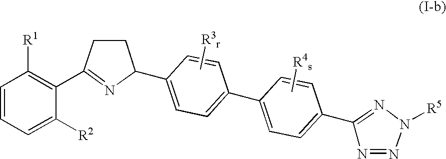
in which
R¹, R², R³, R⁴, R⁵, r and s have the meanings given in any of claims 1 to 5.

7. Δ¹-Pyrrolines of the formula (I-c) 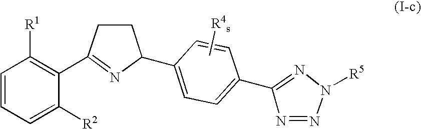
in which
R¹, R², R⁴, R⁵ and s have the meanings given in any of claims 1 to 5.

8. Δ¹-Pyrrolines of the formula (I-d) 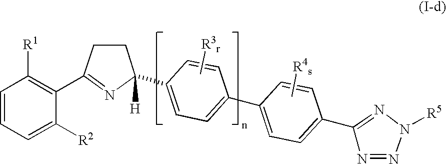
in which
R¹, R², R³, R⁴, R⁵, r and s have the meanings given in any of claims 1 to 5.

9. Δ¹-Pyrrolines of the formula (I-e) 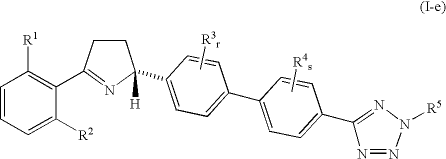
in which
R¹, R², R³, R⁴, R⁵, r and s have the meanings given in any of claims 1 to 5.

10. Δ¹-Pyrrolines of the formula (I-f) 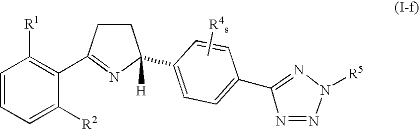
in which
R¹, R², R⁴, R⁵ and s have the meanings given in any of claims 1 to 5.

11. Process for preparing compounds of the formula (I) according to claim 1, characterized in that
A) Δ¹-pyrrolines of the formula (I-a) 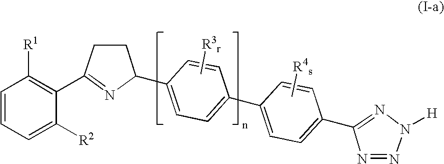
in which
R¹, R², R³, R⁴, n, r and s have the meanings given in claim 1
A1) are reacted with a reagent of the formula (II)
R⁵⁻¹—Z (II)
in which
R⁵⁻¹ represents alkylcarbonyl or represents in each case optionally substituted alkyl or alkylsulphonyl,
Z represents a leaving group,
if appropriate in the presence of a diluent and if appropriate in the presence of an acid binder or
A2) are reacted with an alcohol of the formula (III)
R⁵⁻²—OH (III)
in which
R⁵⁻² represents in each case optionally substituted tertiary alkyl or 1-methyl-cycloalkyl,
in the presence of a strong acid, or
Δ¹-pyrrolines of the formula (I-b) 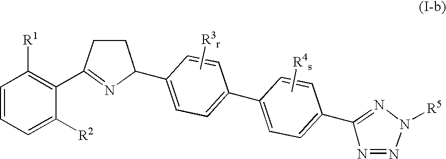
in which
R¹, R², R³, R⁴, R⁵, r and s have the meanings given in claim 1, are obtained by
B) reacting Δ¹-pyrrolines of the formula (IV) 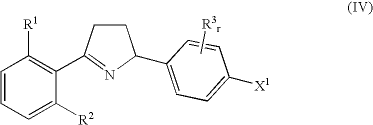
in which
R¹, R², R³ and r have the meanings given in claim 1,
X¹ represents chlorine, bromine, iodine, —OSO₂CF₃ or —OSO₂(CF₂)₃CF₃,
with boron compounds of the formula (V) 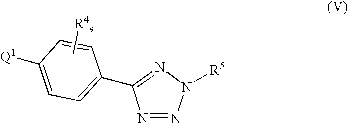
in which
R⁴, R⁵ and s have the meanings given in claim 1 and
Q¹ represents —B(OH)₂, (4,4,5,5-tetramethyl -1,3,2-dioxaborolan)-2-yl, (5,5-dimethyl-1,3,2-dioxaborinan)-2-yl, (4,4,6-trimethyl-1,3,2-dioxaborinan)-2-yl or 1,3,2-benzodioxaborol-2-yl,
in the presence of a catalyst, if appropriate in the presence of an acid binder and if appropriate in the presence of a diluent, or
C) by reacting Δ¹-pyrrolines of the formula (VI) 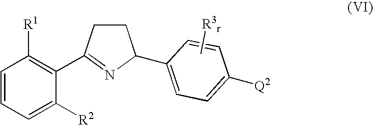
in which
R¹, R², R³ and r have the meanings given in claim 1,
Q² represents —B(OH)₂, (4,4,5,5-tetramethyl-1,3,2-dioxaborolan)-2-yl, (5,5-dimethyl-1,3,2-dioxaborinan)-2-yl, (4,4,6-trimethyl-1,3,2-dioxaborinan)-2-yl or 1,3,2-benzodioxaborol-2-y1 ,
with phenyltetrazoles of the formula (VII) 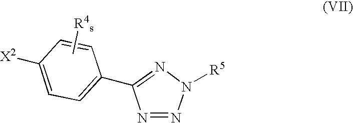
in which
R⁴, R⁵ and s have the meanings given in claim 1 and
X² represents chlorine, bromine, iodine, —OSO₂CF₃ or —OSO₂(CF₂)₃CF₃,
in the presence of a catalyst, if appropriate in the presence of an acid binder and if appropriate in the presence of a diluent, or
D) by reacting Δ¹-pyrrolines of the formula (IV) 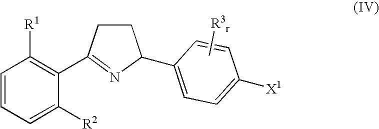
in which
R¹, R², R³ and r have the meanings given in claim 1,
X¹ has the meanings given above,
with phenyltetrazoles of the formula (VII) 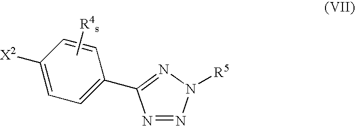
in which
R⁴, R⁵ and s have the meanings given in claim 1,
X² has the meanings given above,
in the presence of a catalyst, in the presence of a diboronic acid ester, if appropriate in the presence of an acid binder and if appropriate in the presence of a diluent in a tandem reaction.

12. Nitriles of the formula (VIII) 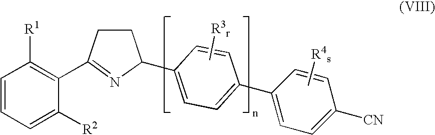
in which
R¹, R², R³, R⁴, n, r and s have the meanings given in any of claims 1 to 5.

13. Aminoketones of the formula (X) 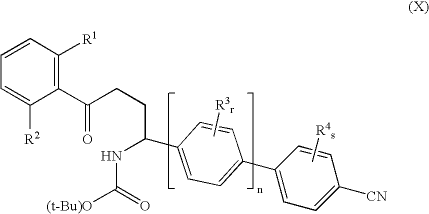
in which
R¹, R², R³, R⁴, n, r and s have the meanings given in any of claims 1 to 5.

14. Lactams of the formula (XI) 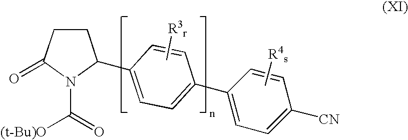
in which
R³, R⁴, n, r and s have the meanings given in any of claims 1 to 5.

15. Lactams of the formula (XIII) 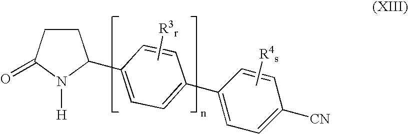
in which
R³, R⁴, n, r and s have the meanings given in any of claims 1 to 5.

16. Pesticide, characterized in that it comprises at least one compound of the formula (I) according to claim 1, in addition to extenders and/or surfactants.

17. Use of compounds of the formula (I) according to claim 1 for controlling pests.

18. Method for controlling pests, characterized in that compounds of the formula (I) according to claim 1 are allowed to act on pests and/or their habitat.

19. Process for preparing pesticides, characterized in that compounds of the formula (I) according to claim 1 are mixed with extenders and/or surfactants.
